(19) United States
(12) Patent Application Publication
(10) Pub. No.: US 2025/0288360 A1
(43) Pub. Date: Sep. 18, 2025

(54) SYSTEMS, METHODS, AND MEDIA FOR DISPLAYING INTERACTIVE EXTENDED REALITY CONTENT

(71) Applicant: Case Western Reserve University, Cleveland, OH (US)

(72) Inventors: Henry Eastman, Cleveland, OH (US); Tianxiang Gao, Cleveland, OH (US); James Gasparatos, Cleveland, OH (US); Robert Gotschall, Cleveland, OH (US); Mark Griswold, Cleveland, OH (US); Erin Henninger, Cleveland, OH (US); Anastasiya Kurylyuk, Cleveland, OH (US); Jeffrey Mlakar, Cleveland, OH (US)

(21) Appl. No.: 18/859,823

(22) PCT Filed: Apr. 25, 2023
(86) PCT No.: PCT/US2023/066200

§ 371 Date: Oct. 24, 2024

Related U.S. Application Data
(60) Provisional application No. 63/334,631, filed on Apr. 25, 2022.

(51) Int. Cl. A61B 34/20; A61B 34/00; A61B 90/00; A61B 90/50

(52) U.S. Cl. CPC A61B 34/20; A61B 34/25; A61B 90/36; A61B 90/50; A61B 2090/365; A61B 2090/502

(57) ABSTRACT

Systems, methods, and media for displaying interactive extended reality content are provided. In some embodiments, a system for presenting extended reality presentations comprises: a transparent display; a plurality of sensors; and at least one processor, wherein the at least one processor is programmed to: receive, via an application program interface (API) server, first content from a first content source; receive, via the API server, second content from a second content source; receive, via the API server, user interface instructions from the second content source; and simultaneously present the first content and the second content in an extended reality presentation in accordance with the user interface instructions.

### CROSS-REFERENCE TO RELATED APPLICATIONS

This application claims the benefit of, and claims priority to U.S. Provisional Application No. 63/334,631, filed Apr. 25, 2022, which is hereby incorporated herein by reference in its entirety for all purposes.

### STATEMENT REGARDING FEDERALLY SPONSORED RESEARCH

N/A

### BACKGROUND

Devices for presenting extended reality (XR) content (e.g., augmented reality (AR) content, mixed reality (MR) content, and/or virtual reality (VR) content) have recently become more prevalent. In general, as described below, XR content is generally incorporated into a compiled application, which limits the content that can be presented to content that was included in the application at the time the application was compiled. Alternatively, an XR application can be configured to dynamically load content (e.g., from a server). However, the user interface of the application and any networking logic needs to be configured to specifically interact with the content dynamically loaded content.

Accordingly, new systems, methods, and media for displaying interactive extended reality content are desirable.

### SUMMARY

In accordance with some embodiments of the disclosed subject matter, systems, methods, and media for displaying interactive extended reality content are provided.

In accordance with some embodiments of the disclosed subject matter, a system for presenting extended reality presentations is provided, the system comprising: a transparent display; a plurality of sensors; and at least one processor, wherein the at least one processor is programmed to: receive, via an application program interface (API) server, first content from a first content source; receive, via the API server, second content from a second content source; receive, via the API server, user interface instructions from the second content source; and simultaneously present the first content and the second content in an extended reality presentation in accordance with the user interface instructions.

In some embodiments, the at least one processor is further programmed to: receive, via the API server, user interface instructions from the first content source; and present the first content in the extended reality presentation in accordance with the user interface instructions received from the first content source.

In some embodiments, the at least one processor is further programmed to: receive, from the API server, a request for information about one or more objects presented in the extended reality presentation; and in response to the request, provide updated information about the one or more objects to the API server.

In some embodiments, the first content source is associated with a first programming language, and the second content source is associated with a different, second programming language.

In some embodiments, the at least one processor is further programmed to: receive, from the API server, updated information about one or more objects presented in the extended reality presentation, wherein at least a first object of the one or more objects is associated with the first content, and a second object of the one or more objects is associated with the second content; and update the extended reality presentation in accordance with the user interface instructions and the updated information.

In some embodiments, the first content source comprises a real-time imaging source, and the second content source comprises an extended reality application configured to facilitate surgery using data from the real-time imaging source.

In accordance with some embodiments of the disclosed subject matter, a system for presenting extended reality presentations is provided, the system comprising: a transparent display; a plurality of sensors; and at least one processor, wherein the at least one processor is programmed to: receive, from a first content source via an application program interface (API) server, instructions to retrieve first content from a first storage location; receive, from a second content source via the API server, instructions to retrieve second content from a second storage location; receive, via the API server, user interface instructions from the second content source; receive, from the first storage location, the first content; receive, from the second storage location, the second content; and simultaneously present the first content and the second content in an extended reality presentation in accordance with the user interface instructions.

In some embodiments, the at least one processor is further programmed to: receive, via the API server, user interface instructions from the first content source; and present the first content in the extended reality presentation in accordance with the user interface instructions received from the first content source.

In some embodiments, the at least one processor is further programmed to: provide updated information about one or more objects presented in the extended reality presentation to the second storage location.

In some embodiments, the first content source is associated with a first programming language, and the second content source is associated with a different, second programming language.

In some embodiments, the at least one processor is further programmed to: receive, from a computing device associated with the second storage location, updated information about one or more objects presented in the extended reality presentation; and update the extended reality presentation in accordance with the user interface instructions and the updated information.

In some embodiments, the first content source comprises a real-time imaging source, and the second content source comprises an extended reality application configured to facilitate surgery using data from the real-time imaging source.

In some embodiments, the first storage location is associated with a cloud storage service.

In some embodiments, the second storage location is associated with a different, second cloud storage service.

In some embodiments, the at least one processor is further programmed to: request, from a computing device associated with the first storage location, updated information about one or more objects presented in the extended reality presentation; request, from a computing device associated with the second storage location, updated information about one or more objects presented in the extended reality presentation; and update the extended reality presentation in accordance with the user interface instructions and the updated information.

In accordance with some embodiments of the disclosed subject matter, a method for presenting extended reality presentations is provided, the method comprising: receiving, via an application program interface (API) server, first content from a first content source; receive, via the API server, second content from a second content source; receive, via the API server, user interface instructions from the second content source; and simultaneously present the first content and the second content in an extended reality presentation in accordance with the user interface instructions.

In accordance with some embodiments of the disclosed subject matter, a method for presenting extended reality presentations is provided, the method comprising: receiving, from a first content source via an application program interface (API) server, instructions to retrieve first content from a first storage location; receiving, from a second content source via the API server, instructions to retrieve second content from a second storage location; receiving, via the API server, user interface instructions from the second content source; receiving, from the first storage location, the first content; receiving, from the second storage location, the second content; and simultaneously presenting the first content and the second content in an extended reality presentation in accordance with the user interface instructions.

In accordance with some embodiments of the disclosed subject matter, a non-transitory computer-readable medium storing computer-executable code, comprising code for causing a computer to cause a processor to perform a method for presenting extended reality presentations is provided, the method comprising: receiving, via an application program interface (API) server, first content from a first content source; receive, via the API server, second content from a second content source; receive, via the API server, user interface instructions from the second content source; and simultaneously present the first content and the second content in an extended reality presentation in accordance with the user interface instructions.

In accordance with some embodiments of the disclosed subject matter, a non-transitory computer-readable medium storing computer-executable code, comprising code for causing a computer to cause a processor to perform a method for presenting extended reality presentations is provided, comprising: receiving, from a first content source via an application program interface (API) server, instructions to retrieve first content from a first storage location; receiving, from a second content source via the API server, instructions to retrieve second content from a second storage location; receiving, via the API server, user interface instructions from the second content source; receiving, from the first storage location, the first content; receiving, from the second storage location, the second content; and simultaneously presenting the first content and the second content in an extended reality presentation in accordance with the user interface instructions.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1 shows an example of a head mounted display that can be used in accordance with some embodiments of the disclosed subject matter.

FIG. 2 shows an example of a system of networked extended reality devices in accordance with some embodiments of the disclosed subject matter.

FIG. 3 shows an example of hardware that can be used to implement at least one head mounted display, at least one server, and at least one user input device in accordance with some embodiments of the disclosed subject matter.

FIG. 4A shows an example of an information flow among a content source device uploading content and/or user interface instructions to a server, and an extended reality device receiving the content and/or user interface instructions and presenting the content in accordance with the user interface instructions in accordance with some embodiments of the disclosed subject matter.

FIG. 4B shows another example of an information flow among a content source device uploading content and/or user interface instructions to a server, and an extended reality device receiving the content and/or user interface instructions and presenting the content in accordance with the user interface instructions in accordance with some embodiments of the disclosed subject matter.

FIG. 5A shows an example of a process for providing content and user interface instructions to one or more extended reality devices in accordance with some embodiments of the disclosed subject matter.

FIG. 5B shows another example of a process for providing content and user interface instructions to one or more extended reality devices in accordance with some embodiments of the disclosed subject matter.

FIG. 6 shows an example of two conventional extended reality applications that include content and user interface instructions.

FIG. 7 shows an example, of two conventional networked extended reality applications that include content and user interface instructions used to present the content.

FIG. 8 shows an example of two conventional networked extended reality applications configured to dynamically load content and use included user interface instructions to present the dynamically loaded content.

FIG. 9A shows an example of an extended reality device executing an extended reality presentation application configured to receive content and user interface instructions from an extended reality source via an extended reality presentation server and present the content based on the user interface instructions in accordance with some embodiments of the disclosed subject matter.

FIG. 9B shows an example of an extended reality device executing an extended reality presentation application configured to receive content and user interface instructions from an extended reality source via an extended reality presentation server and present the content based on the user interface instructions in accordance with some embodiments of the disclosed subject matter.

FIG. 10A shows an example of multiple extended reality devices executing extended reality presentation applications configured to receive content and user interface instructions from an extended reality source via an extended reality presentation server and present the content based on the user interface instructions in accordance with some embodiments of the disclosed subject matter.

FIG. 10B shows another example of multiple extended reality devices executing extended reality presentation applications configured to receive user interface instructions from an extended reality source via an extended reality presentation server, receive content from a content storage locations specified by the extended reality source, and present the content based on the user interface instructions in accordance with some embodiments of the disclosed subject matter.

FIG. 11 shows an example of multiple different types of extended reality devices executing extended reality presentation applications configured to receive content and user interface instructions from an extended reality source via an extended reality presentation server and present the content based on the user interface instructions in accordance with some embodiments of the disclosed subject matter.

FIG. 12A shows an example of an extended reality device executing an extended reality presentation application configured to receive content and user interface instructions from extended reality sources via an extended reality presentation server, and multiple extended reality sources configured to provide content to the extended reality device in accordance with some embodiments of the disclosed subject matter.

FIG. 12B shows another example of an extended reality device executing an extended reality presentation application configured to receive content and user interface instructions from extended reality sources via an extended reality presentation server, and multiple extended reality sources configured to provide content to the extended reality device in accordance with some embodiments of the disclosed subject matter.

FIG. 13A shows an example of an extended reality device executing an extended reality presentation application configured to receive content and user interface instructions from extended reality sources via an extended reality presentation server, and multiple extended reality sources implemented in different programming languages configured to provide content to the extended reality device in accordance with some embodiments of the disclosed subject matter.

FIG. 13B shows another example of an extended reality device executing an extended reality presentation application configured to receive content and user interface instructions from extended reality sources via an extended reality presentation server, and multiple extended reality sources implemented in different programming languages configured to provide content to the extended reality device in accordance with some embodiments of the disclosed subject matter.

FIG. 14A shows a more particular example, of two conventional networked extended reality applications that include content and user interface instructions used to present the content.

FIG. 14B shows an example of multiple extended reality devices executing extended reality presentation applications configured to receive content and user interface instructions from an extended reality source via an extended reality presentation server, and an extended reality source configured to provide multiple sets of content to the extended reality devices in accordance with some embodiments of the disclosed subject matter.

FIG. 14C shows an example of multiple extended reality devices executing extended reality presentation applications configured to receive content and user interface instructions from an extended reality source via an extended reality presentation server, and multiple extended reality sources configured to provide different sets of content to the extended reality devices in accordance with some embodiments of the disclosed subject matter.

FIG. 14D shows another example of multiple extended reality devices executing extended reality presentation applications configured to receive content and user interface instructions from an extended reality source via an extended reality presentation server, and multiple extended reality sources configured to provide different sets of content to the extended reality devices in accordance with some embodiments of the disclosed subject matter.

FIG. 15A shows an example of a conventional extended reality application configured to dynamically load content from multiple sources, and present extended reality content based on the multiple sources of content.

FIG. 15B shows an example of an extended reality device executing an extended reality presentation application configured to receive content and user interface instructions from one or more extended reality sources via an extended reality presentation server, and multiple extended reality sources configured to provide content that can be dynamically loaded by the extended reality device and presented in an extended reality experience based on the multiple sources of content in accordance with some embodiments of the disclosed subject matter.

FIG. 15C shows another example of an extended reality device executing an extended reality presentation application configured to receive content and user interface instructions from one or more extended reality sources via an extended reality presentation server, and multiple extended reality sources configured to provide content that can be dynamically loaded by the extended reality device and presented in an extended reality experience based on the multiple sources of content in accordance with some embodiments of the disclosed subject matter.

### DETAILED DESCRIPTION

Before any embodiments of the disclosed subject matter are explained in detail, it is to be understood that the disclosed subject matter is not limited in its application to the details of construction and the arrangement of components set forth in the following description or illustrated in the following drawings. The disclosed subject matter is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the disclosed subject matter. Various modifications to the illustrated embodiments will be readily apparent to those skilled in the art, and the generic principles herein can be applied to other embodiments and applications without departing from embodiments of the disclosed subject matter. Thus, embodiments of the disclosed subject matter are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the disclosed subject matter. Skilled artisans will recognize the examples provided herein have many useful alternatives and fall within the scope of embodiments of the disclosed subject matter.

In accordance with some embodiments of the disclosed subject matter, mechanisms (which can include systems, methods and/or media) for displaying interactive extended reality content are provided. In some embodiments, an extended reality presentation application can be executed by an extended reality device, and the extended reality presentation application can be configured to receive user interface instructions and/or content from one or more other applications (e.g., being executed remotely and/or locally) and/or one or more sources of content (e.g., remote and/or local sources of content).

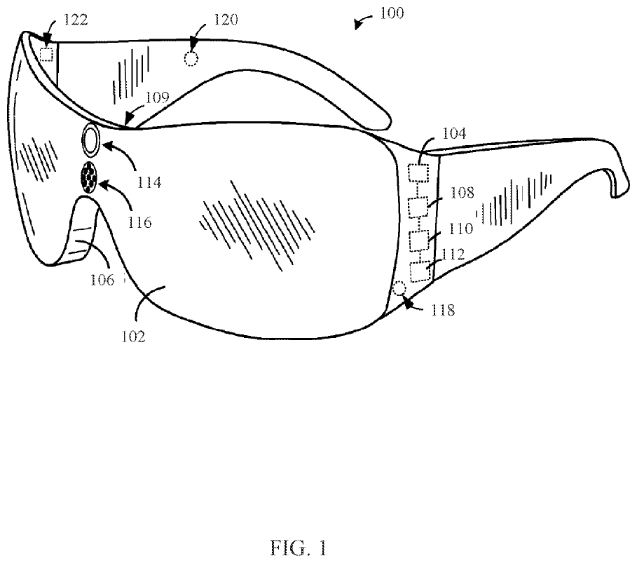

FIG. 1 shows an example 100 of a head mounted display (HMD) that can be used in accordance with some embodiments of the disclosed subject matter. As shown in FIG. 1, head mounted display 100 can include a display processor 104 and a transparent display 102 that can be used to present images, such as holographic objects, to the eyes of a wearer of HMD 100. In some embodiments, transparent display 102 can be configured to visually augment an appearance of a physical environment to a wearer viewing the physical environment through transparent display 102. For example, in some embodiments, the appearance of the physical environment can be augmented by graphical content (e.g., one or more pixels each having a respective color and brightness) that is presented via transparent display 102 to create a mixed reality (or augmented reality environment). Note that as used herein, mixed reality and augmented reality are meant to convey similar experiences, but a mixed reality environment is intended to convey a more immersive environment than an augmented reality environment. Additionally or alternatively, in some embodiments, transparent display 102 can be configured to render a fully opaque virtual environment (e.g., by using one or more techniques to block the physical environment from being visible through HMD 100). In some embodiments, a non-transparent display can be used as a transparent display 102. In some such embodiments, one or more cameras can be used to generate a real-time representation of at least a portion of the physical environment in which HMD 100 is located. For example, an HMD with a non-transparent display can simulate a mixed reality environment using images of a physical environment and graphics (e.g., 3D models) displayed with the images of the physical environment as though the graphics are physically present within the physical environment. Accordingly, as used herein, transparent display 102 can include both transparent displays that visually augment an appearance of a physical environment to a wearer viewing the physical environment through transparent display, and non-transparent displays that visually augment an appearance of a physical environment to a wearer viewing the physical environment as images captured by external cameras and presented by the display to simulate viewing of the physical environment. In some such embodiments, HMD 100 can be used to present a virtual reality environment. In some embodiments, HMD 100 can be configured as a device to present extended reality (XR) content. Examples of XR content can include augmented reality (AR) content, mixed reality (MR) content, and/or virtual reality (VR) content. As described below, other types of devices (e.g., mobile devices, such as smartphones, tablet computers, etc.) can be used to present XR content.

As shown in FIG. 1, in some embodiments, transparent display 102 can include one or more image producing elements (e.g., display pixels) located within lenses 106 (such as, for example, pixels of a see-through Organic Light-Emitting Diode (OLED) display). Additionally or alternatively, in some embodiments, transparent display 102 can include a light modulator on an edge of the lenses 106.

In some embodiments, HMD 100 can include various sensors and/or other related systems. For example, HMD 100 can include a gaze tracking system 108 that can include one or more image sensors that can generate gaze tracking data that represents a gaze direction of a wearer's eyes. In some embodiments, gaze tracking system 108 can include any suitable number and arrangement of light sources and/or image sensors. For example, as shown in FIG. 1, the gaze tracking system 108 of HMD 100 can utilize at least one inward facing sensor 109. In some embodiments, a user can be prompted to permit the acquisition and use of gaze information to track a position and/or movement of the user's eyes.

In some embodiments, HMD 100 can include a head tracking system 110 that can utilize one or more motion sensors, such as motion sensors 112 shown in FIG. 1, to capture head pose data that can be used to track a head position of the wearer, for example, by determining the direction and/or orientation of a wearer's head. In some embodiments, head tracking system 110 can include an inertial measurement unit configured as a three-axis or three-degree of freedom position sensor system.

In some embodiments, head tracking system 110 can also support other suitable positioning techniques, such as Global Positioning System (GPS) or other global navigation systems, indoor position tracking systems (e.g., using Bluetooth low energy beacons), etc. Further, while specific examples of position sensor systems have been described, it will be appreciated that any other suitable position sensor systems can be used. For example, head pose and/or movement data can be determined based on sensor information from any suitable combination of sensors mounted on the wearer and/or external to the wearer including but not limited to any number of gyroscopes, accelerometers, inertial measurement units (IMUs), GPS devices, barometers, magnetometers, cameras (e.g., visible light cameras, infrared light cameras, time-of-flight depth cameras, structured light depth cameras, etc.), communication devices (e.g., Wi-Fi antennas/interfaces, Bluetooth, etc.), etc.

In some embodiments, HMD 100 can include an optical sensor system that can utilize one or more outward facing sensors, such as optical sensor 114, to capture image data of the environment. In some embodiments, the captured image data can be used to detect movements captured in the image data, such as gesture-based inputs and/or any other suitable movements by a user waring HMD 100, by another person in the field of view of optical sensor 114, or by a physical object within the field of view of optical sensor 114. Additionally, in some embodiments, the one or more outward facing sensor(s) can capture 2D image information and/or depth information from the physical environment and/or physical objects within the environment. For example, the outward facing sensor(s) can include a depth camera, a visible light camera, an infrared light camera, a position tracking camera, and/or any other suitable image sensor or combination of image sensors.

In some embodiments, a structured light depth camera can be configured to project a structured infrared illumination, and to generate image data of illumination reflected from a scene onto which the illumination is projected. In such embodiments, a depth map of the scene can be constructed based on spacing between features in the various regions of an imaged scene. Additionally or alternatively, in some embodiments, a continuous wave time-of-flight depth camera, a pulsed time-of-flight depth camera or other sensor (e.g., LiDAR), etc. In some embodiments, illumination can be provided by an infrared light source 116, and/or a visible light source.

In some embodiments, the HMD 100 can include a microphone system that can include one or more microphones, such as microphone 118, which can capture audio data. In some embodiments, audio can be presented to the wearer via one or more speakers, such as speaker 120.

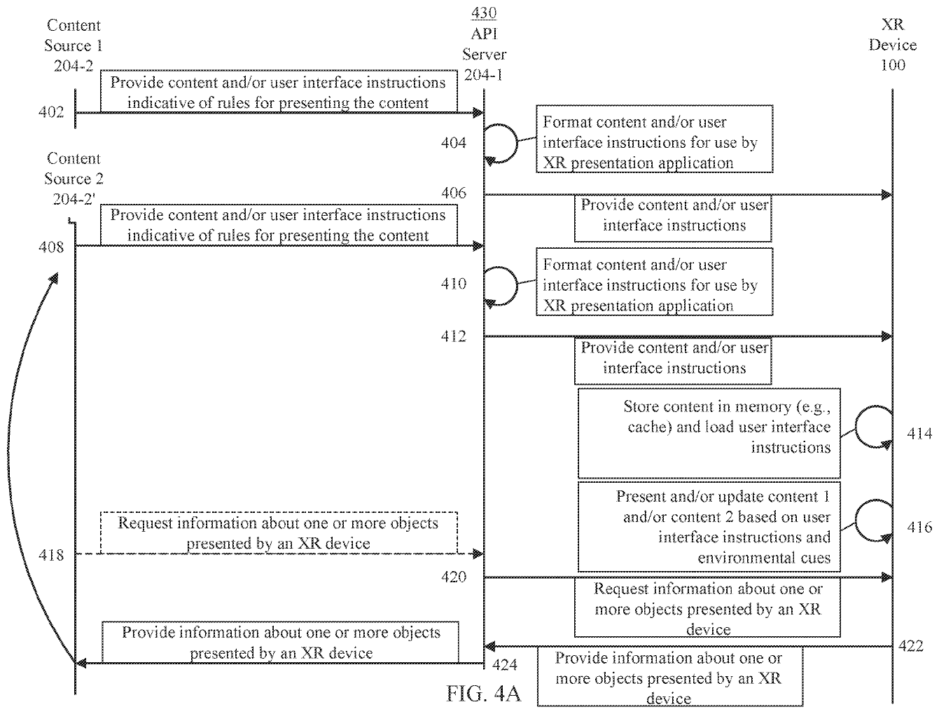

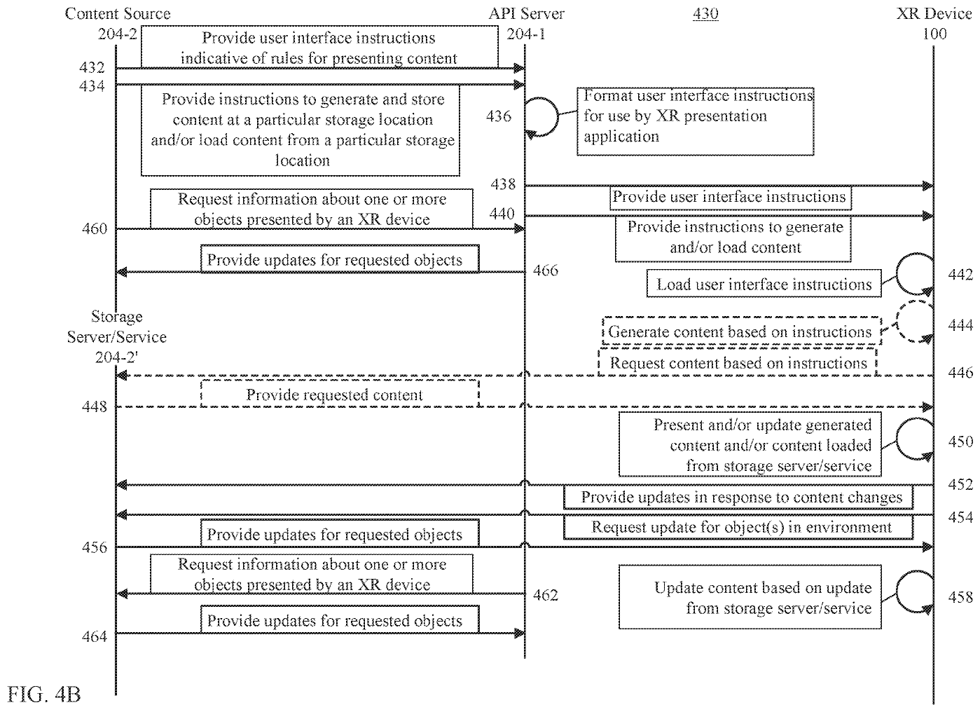

In some embodiments, HMD 100 can include a controller, such as controller 122, which can include, for example, a processor and/or memory (as described below in connection with FIGS. 4A and 4B) that are in communication with the various sensors and systems of HMD 100. In some embodiments, controller 122 can store, in memory, instructions that are executable by the processor to receive signal inputs from the sensors, determine a pose of HMD 100, and adjust display properties for content displayed using transparent display 102.

In some embodiments, HMD 100 can have any other suitable features or combination of features, such as features described in U.S. Pat. No. 9,495,801 issued to Microsoft Technology Licensing, LLC, which is hereby incorporated by reference herein in its entirety. The description herein of HMD 100 is merely for illustration of hardware that can be used in connection with the disclosed subject matter. However, the disclosed subject matter can be used with any suitable mixed reality device and/or augmented reality device, such as the HoloLens® made by Microsoft®, and/or devices described in U.S. Pat. Nos. 8,847,988, 8,941,559, and U.S. Patent Application Publication No. 2014/0160001, each of which is hereby incorporated by reference herein in its entirety.

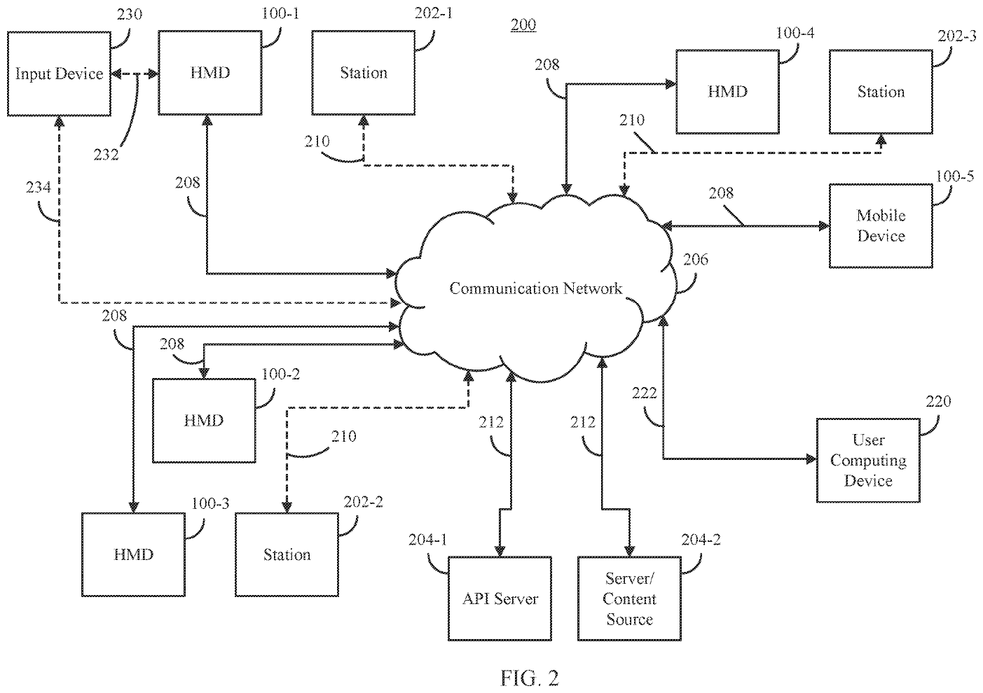

FIG. 2 shows an example 200 of a system of networked extended reality devices 100 in accordance with some embodiments of the disclosed subject matter. As shown in FIG. 2, system 200 can include various XR devices 100-1 to 100-5 located in the same physical space (e.g., in the same room). System 200 can include various predefined physical anchor points 202-1 to 202-3 (which are sometimes referred to herein as pods or stations), which can correspond to points in physical space at which one or more images are to be displayed by XR devices 100. For example, each station 202 can be used by HMDs 100 as a predetermined spatial anchor for one or more images. In a more particular example, stations 202 can define spatial anchors for images to be displayed by an XR device (e.g., HMD 100) if the XR device is instructed to present an image corresponding to the station as long as the station is in the field of view, even if the XR device moves relatively far from the station (e.g., more than five meters from the station). In another more particular example, stations 202 can define spatial anchors for images to be displayed by an XR device (e.g., HMD 100) if the XR device is instructed to present an image corresponding to a particular station or any station that satisfies one or more criteria (e.g., based on distance, filed of view, etc.), even if the XR device moves relatively far from the stations (e.g., more than five meters from the station). In some embodiments, content be anchored at physical locations in the environment that satisfy a particular criterion or criteria. Such a location may or may not correspond to a station (e.g., the location may correspond to any flat surface, to a particular anatomical structure, etc.).

In some embodiments, system 200 can include one or more servers 204 that can receive (e.g., via an API) content and/or user interface instructions to be used by one or more XR devices. Additionally, in some embodiments, system 200 can include one or more servers 204 that can provide and/or generate content, and/or user interface instructions that can be provided to the one or more XR devices (e.g., via an API server). In some embodiments, server 204 can be implemented using any suitable computing device such as a server computer, an HMD, a tablet computer, a smartphone, a personal computer, a laptop computer, etc. In some embodiments, each XR device 100 can connect to communication network 206 via a communications link 208, and server(s) 204 can connect to communication network 206 via a communications link 212. In some such embodiments (e.g., embodiments in which stations 202 are active devices), stations 202 can connect to communication network 206 via a communications link 210. In some embodiments, a user computing device 220 can connect to communication network 206 via a communications link 222. Communication network 206 can be any suitable communication network or combination of communication networks. For example, communication network 206 can be a Wi-Fi network (which can include one or more wireless routers, one or more switches, etc.), a peer-to-peer network (e.g., a Bluetooth network, a Zigbee mesh network, etc.), a cellular network (e.g., a 3G network, a 4G network, a 5G network, etc., complying with any suitable standard, such as CDMA, GSM, LTE, LTE Advanced, WiMAX, NR, etc.), a wired network, etc. Communications links 208, 210 and 212 can each be any suitable communications link or combination of communications links, such as a Wi-Fi links, Bluetooth links, cellular links, etc.

In some embodiments, an API server 204-1 can be configured to receive (e.g., via an API) content (e.g., XR content) and/or user interface instructions from one or more sources (e.g., one or more devices executing an XR content application, one or more devices configured to provide data that can be used to control an XR experience, one or more devices configured to provide content that can be used to as part of an XR experience, etc.). In some embodiments, multiple API servers 204-1 can be implemented and used (e.g., with different RX devices). In some embodiments, actions described herein as being performed by API server 204-1 can be implemented by an XR device.

In some embodiments, a content server 204-2 can be configured to provide content and/or user interface instructions (e.g., associated with an XR content application). For example, content server 204-2 can be configured to transmit content and/or user interface instructions to API server 204-1 (e.g., in response to a request from a computing device, such as XR device 100).

Additionally or alternatively, in some embodiments, content server 204-2 can be configured to store content and/or properties of content that can be provided to HMDs 100 and/or other user computing devices participating in and/or controlling an XR content presentation (e.g., including content from multiple different sources, user interfaces from multiple different sources, including different HMDs and/or other XR devices that have different operating systems, etc.). For example, in some embodiments, content server 204-2 can be implemented as using a dedicated content server (e.g., operating separately from API server 204-1, though both can operate on the same physical server computer, for example, as virtual machines). As another example, in some embodiments, content server 204-2 can be implemented using a data storage service (e.g., cloud-based storage), which can be serverless. In a more particular example, services such Blob Storage provided by Microsoft Azure, file Dropbox file hosting provided by Dropbox, Inc., Amazon Simple Storage Service (S3) object storage service provided by Amazon Web Services, Inc., can be used to store objects and/or other content (and/or properties associated with an object and/or other content, such as scale/size, shape, location, orientation, color(s), textures, etc.). As described below, in some embodiments, content server 204-2 can maintain a record of current properties of an object, and HMDs (and/or other computing devices) participating in an XR presentation can request the properties and update objects in the XR presentation based on a response from content server 204-2.

In some embodiments, a user can interact with content server 204-2 via user computing device 220 to provide user interface instructions and/or select content that is to be presented in connection with each station 202. For example, the user can create a program in any suitable programming language (e.g., Python, R, C #, C++, etc.). In such an example, the program can include user interface instructions and/or content to be presented in by an XR device (e.g., in an AR, MR, and/or VR experience).

As another example, in some embodiments, a user can interact with content server 204-2 via user computing device 220 to instruct content server 204-1 to cause XR devices in proximity to station 202-2 to present images showing an interactive 3D model of the human vascular system in the absence of certain other anatomical features (e.g., in the absence of muscles, in the absence of the skeletal system, etc.), while the user can instruct content server 204 to cause XR devices in proximity to station 202-3 to present images showing an interactive 3D model showing how the vascular system integrates into certain major muscle groups.

In some embodiments, the user can generate user interface instructions that control how content associated with a particular XR content application is to be presented in connection with one or more stations 202 and/or environmental cues via user computing device 220 and/or content server 204-2. Such user interface instructions can include, for example, which content is to be presented in connection with particular physical locations and/or environmental cues, times (e.g., a length of time, a period of time during the presentation, etc.) at which the content is to be presented, an order in which content is to be presented at each station (and/or across all stations), actions to be performed in response to user interaction with the content and/or portions of the content, etc.

In some embodiments, content and/or user interface instructions (as described below, the content and/or user interface instructions can be encrypted) can be conveyed by content server 204-2 to each XR device 100 (e.g., via API server 204-1) at the time of the presentation as a series of data objects (e.g., JavaScript Object Notation (JSON) objects), as a document (e.g., an XML document), in a custom format (e.g., binary coded instructions) etc., referencing the content that is to be presented, the order in which it is to be presented, a time(s) at which it is to be presented, actions to be performed in response to interaction with the content and/or a portion of the content, etc. For example, content server 204-2 can send instructions (e.g., an XML file, one or more JSON objects, one or more binary coded instructions, etc.) that an XR device 100 can use to present content provided by content server 204-2. In some embodiments, user interface instructions provided by a content server 204-2 can be formatted as a change(s) from a previous state (e.g., an original state, a state at a particular time, etc.). For example, user interface instructions provided by content server 204-2 can provide instructions that are a change from the previous state. In some embodiments, content server 204-2 can periodically (e.g., at regular and/or irregular intervals) provide all user interface instructions, which can be used as a state against which to evaluate new instructions. In some embodiments, content server 204-2 can cause XR devices participating in a presentation to retrieve content to be rendered in an XR presentation from one or more particular locations (e.g., one or more storage servers and/or storage services) (e.g., using an instruction sent via API server 204-1) at the time of the presentation as a series of data objects (e.g., JavaScript Object Notation (JSON) objects), as a document (e.g., an XML document), in a custom format (e.g., binary coded instructions) etc., referencing the content that is to be retrieved and/or presented. In some embodiments, content server 204-2 can provide user interface instructions and pointers to content to be retrieved by XR devices. Additionally or alternatively, the user interface instructions can include instructions to create a certain object(s) (e.g., primitive objects) with particular properties, and refer to a particular storage location to maintain updated properties associated with the object that has been created (e.g., as the object is manipulated by users of XR devices participating in the XR presentation).

In some embodiments, user computing device 220 can be any suitable computing device or combination of devices, such as a personal computer, a laptop computer, a tablet computer, a smartphone, a wearable computer, a head mounted display, etc. In some embodiments, a user can select content, generate user interface instructions, upload content, etc., using user computing device 220 and/or content server 204-2 using any suitable technique or combination of techniques. For example, user computing device 220 can execute a development application from memory that is configured to facilitate generation and/or editing of a presentation application for any suitable XR experience and/or any suitable number of XR devices. As another example, user computing device 220 can interact with a development application executed by another computing device (e.g., server 204, a cloud server, etc.) through network 206 (e.g., via a web browser executed by computing device 220 or other application that facilitates interaction with a remotely executed development application). In some embodiments, user computing device 220 can be used to control at least a portion of an ongoing XR presentation. For example, at least a portion of the XR presentation can be presented (e.g., using a 2D display, such as a computer monitor or touchscreen). In some embodiments, user computing device 220 can provide user interface instructions to each XR device 100 (e.g., via API server 204-1) at the time of the presentation, and/or can cause XR devices participating in a presentation to retrieve content to be rendered in an XR presentation from one or more particular locations (e.g., one or more storage servers and/or storage services) (e.g., using an instruction sent via API server 204-1) at the time of the presentation.

In some embodiments, each XR device 100 can execute an application that can interact with API server 204-1 (e.g., over communication network 206) to receive (and/or retrieve) content and/or user interface instructions associated with a particular XR content application (e.g., provided via different content servers 204-2).

In some embodiments, system 200 can determine which content is to be presented by a particular XR device 100 using any suitable technique or combination of techniques. For example, XR device 100 can determine which content to present based on user interface instructions received from one or more XR content applications, based on environmental cues, based on content and/or user interface instructions received from another XR content application, based on content received from a content source that is not executing an XR content application. In such an example, XR device 100 can use any suitable technique to determine which content to present and/or how to present the content, such as by analyzing image data captured by an outward facing camera (e.g., optical sensor 114) for environmental cues, analyzing the strength of various signals (e.g., Bluetooth signals) to determine environmental cues, analyzing GPS coordinates of XR device 100 determined using a GPS receiver, etc. As another example, XR device 100 can provide information (e.g., one or more images, signal strength of various signals, GPS coordinates, etc.) to a server (e.g., content server 204-2, a presentation server), which can determine which content is to be presented by XR device 100.

As still another example, XR device 100 can determine an environmental cue indicative of which content is to be presented, and can request content associated with that environmental cue from content server 204-2 and/or can present content associated with that environmental cue from memory.

In some embodiments, XR device 100 can determine that different content is to be presented at any suitable time (e.g., based on user interface instructions received from one or more XR content applications and/or other content sources).

In some embodiments, API sever 204-1, content server 204-2, and/or any other suitable server can communicate additional information to XR devices 100 during presentation of content, such as instructions for one or more of XR devices 100 about how to present content and/or additional content to be presented. For example, a user of a first XR device 100-1 can use an input device (e.g., a dedicated user interface device, an optical sensor used to capture images of a user's hand, etc.) to point (e.g., via a line through space, a dot on the content, the user's hand, etc.) to a particular portion of content being presented by XR device 100-1, and a server can send instructions to one or more other XR devices 100 presenting the same content (e.g., locally and/or remotely, in the same physical environment or a different physical or virtual environment, etc.) that causes each of those XR devices to present supplemental content showing that the user of XR device 100-1 is pointing to a particular portion of the content. In some embodiments, such additional information can be used to control presentation of content by XR devices 100. For example, a user of XR 100-1 can control a presentation via input to XR device 100-1 (and/or any other suitable device), and one or more other XR device 100 can receive instructions and/or content from a server (e.g., API server 204-1) that cause the one or more other XR devices 100 to change which content is being presented in accordance with the input from the user of XR device 100-1.

In some embodiments, audio information can be recorded (e.g., by XR device 100) and/or communicated (e.g., to one or more other XR devices 100 participating in a particular shared XR experience), which can be presented in connection with the content by XR device 100. Additionally or alternatively, in some embodiments, audio can be recorded by another device (e.g., hardware that is part of station 202 and/or by microphone 118 of one or more XR devices 100). In some embodiments, audio can be recorded at the request of the wearer of a particular XR device 100 for later access (e.g., as a study aid).

Although XR devices 100-1 to 100-5 are described above as being local to each other (e.g., in the same room), XR devices in system 200 can be located local to each other and/or remote from each other. For example, system 200 can be used to collaborate and/or interact with one or more users of XR devices 100 located in one or more remote locations. In some embodiments, two XR devices 100 can be remote from each other if there is not a line of sight between them. For example, two XR devices 100 can be considered remote from each other if they are located in different rooms, regardless of whether they are both connected to the same local area network (LAN) or to different networks. As another example, two XR devices 100 that are connected to different LANs can be considered remote from each other. As yet another example, two XR devices 100 that are connected to different subnets can be considered remote from each other. As still another example, two XR devices 100 that are configured to present different types of XR content (e.g., MR and VR, respectively, can be considered remote from each other regardless of whether the devices are located in the same physical environment. In some embodiments, two XR devices 100 that are remote from each other can be used to collaborate by representing a remote user with an avatar in connection with a hologram being presented by at least one of the two XR devices 100.

In some embodiments, API server 204-1 and/or content server 204-2 can be located locally or remotely from XR devices 100. Additionally, in some embodiments, multiple API server 204-1 and/or content servers 204-2 can be used (which may be located in different physical locations) to provide different content, provide redundant functions, etc. In some embodiments, one of the XR devices 100 in system 200 can perform one or more of the operations of API server 204-1 and/or one or more content servers 204-2 described herein, such as providing user interface instructions to an XR presentation application being executed by the XR device 100 and/or other XR devices. For example, local XR devices 100 in system 200 can be interconnected to form a mesh network, and an XR device acting as API server 204-1 (e.g., XR device 100-1) can provide user interface instructions and/or content from one or more XR content applications. Additionally, in some embodiments, the XR device acting as API server 204-1 can be a node in the mesh network, and can communicate over another network (e.g., a LAN, cellular, etc.) to receive other information, such as information related to a remote user.

In some embodiments, input device 230 can communicate with XR device 100-1 via a communications link 232. In some embodiments, communications link 232 can be any suitable communications link that can facilitate communication between input device 230 and XR device 100-1. For example, communications link 232 can be a wired link (e.g., a USB link, an Ethernet link, a proprietary wired communication link, etc.) and/or a wireless link (e.g., a Bluetooth link, a Wi-Fi link, etc.). In some embodiments, input device 230 can include any suitable sensors for determining a position of input device 230 with respect to one or more other devices and/or objects (e.g., XR device 100-1, station 202, a particular body part of a wearer of XR device 100-1, a particular portion of an environment, etc.), and/or a relative change in position (e.g., based on sensor outputs indicating that input device 230 has been accelerated in a particular direction, that input device 230 has been rotated in a certain direction, etc.). For example, in some embodiments, input device 230 can include one or more accelerometers, one or more gyroscopes, one or more electronic compasses, one or more image sensors, an inertial measurement unit, etc. In some embodiment, in addition to or in lieu of communication link 232, input device 230 can communicate with XR device 100-1, API server 204-1, and/or any other suitable device(s) via a communication link 234. In some embodiments, communication link 234 can be any suitable communications link or combination of communications links, such as a Wi-Fi link, a Bluetooth link, a cellular link, etc.

In some embodiments, input device 230 can be a separate device that can convey location information to XR device 100-1 and/or API server 204-1, which can then be used to control one or more portions of a user interface.

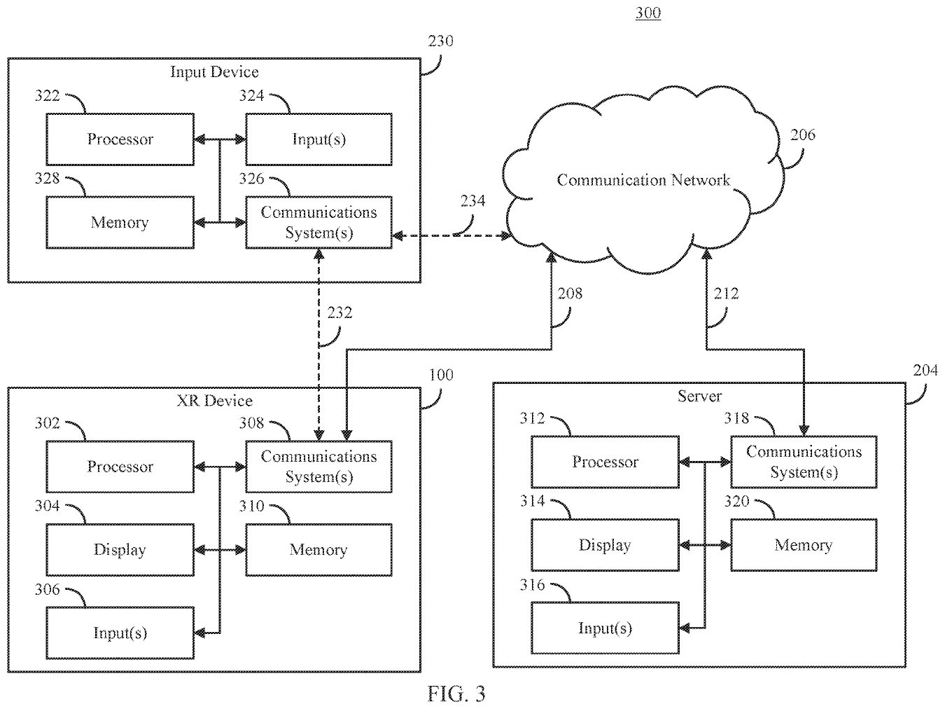

FIG. 3 shows an example 300 of hardware that can be used to implement at least one of XR device 100, server 204 and input device 230 in accordance with some embodiments of the disclosed subject matter. As shown in FIG. 3, in some embodiments, XR device 100 can include a processor 302, a display 304, one or more inputs 306, one or more communication systems 308, and/or memory 310. In some embodiments, processor 302 can be any suitable hardware processor or combination of processors, such as a central processing unit (CPU), a graphics processing unit (GPU), an accelerated processing unit (APU), etc. In some embodiments, display 304 can include any suitable display device(s), such as a transparent display as described above in connection with FIG. 1, a non-transparent 3D display (e.g., a VR display), a 2D display (e.g., a touchscreen of a smartphone, a touchscreen of a tablet computer, etc.). In some embodiments, inputs 306 can include any suitable input device(s) and/or sensor(s) that can be used to receive user input, such as gaze tracking system 108, head tracking system 110, motion sensors 112, optical sensor 114, microphone 118, etc.

In some embodiments, communications systems 308 can include any suitable hardware, firmware, and/or software for communicating information over communication network 206 and/or any other suitable communication networks. For example, communications systems 308 can include one or more transceivers, one or more communication chips and/or chip sets, etc. In a more particular example, communications systems 308 can include hardware, firmware and/or software that can be used to establish a Wi-Fi connection, a Bluetooth connection, a cellular connection, etc.

In some embodiments, memory 310 can include any suitable storage device or devices that can be used to store instructions, values, etc., that can be used, for example, by processor 302 to present content using display 304, to communicate with server 204 via communications system(s) 308, etc. Memory 310 can include any suitable volatile memory, non-volatile memory, storage, any other suitable type of storage medium, or any suitable combination thereof. For example, memory 310 can include RAM, ROM, EEPROM, one or more flash drives, one or more hard disks, one or more solid state drives, one or more optical drives, etc. In some embodiments, memory 310 can have encoded thereon a computer program for controlling operation of XR device 100. In some such embodiments, processor 302 can execute at least a portion of the computer program to present content (e.g., one or more holograms), receive content from content server 204-2 and/or API server 204-1, transmit information to content server 204-2 and/or API server 204-1, etc. In some embodiments, XR device 100 can use any suitable hardware and/or software for rendering the content received from server 204, such as Unity 3D available from Unity Technologies. Additionally, in some embodiments, any suitable communications protocols can be used to communicate control data, image data, audio, etc., between XR device 100 and one or more servers 204, such networking software available from Unity Technologies.

In some embodiments, server 204 can include a processor 312, a display 314, one or more inputs 316, one or more communication systems 318, and/or memory 320. In some embodiments, processor 312 can be any suitable hardware processor or combination of processors, such as a CPU, a GPU, an APU, etc. In some embodiments, display 314 can include any suitable display devices, such as a computer monitor, a touchscreen, a television, etc. In some embodiments, inputs 316 can include any suitable input devices and/or sensors that can be used to receive user input, such as a keyboard, a mouse, a touchscreen, a microphone, etc.

In some embodiments, communications systems 318 can include any suitable hardware, firmware, and/or software for communicating information over communication network 306 and/or any other suitable communication networks. For example, communications systems 318 can include one or more transceivers, one or more communication chips and/or chip sets, etc. In a more particular example, communications systems 318 can include hardware, firmware and/or software that can be used to establish a Wi-Fi connection, a Bluetooth connection, a cellular connection, etc.

In some embodiments, memory 320 can include any suitable storage device or devices that can be used to store instructions, values, etc., that can be used, for example, by processor 312 to present content using display 314, to communication with one or more XR device 100, etc. Memory 320 can include any suitable volatile memory, non-volatile memory, storage, any other suitable type of storage medium, or any suitable combination thereof. For example, memory 320 can include RAM, ROM, EEPROM, one or more flash drives, one or more hard disks, one or more solid state drives, one or more optical drives, etc. In some embodiments, memory 320 can have encoded thereon a server program for controlling operation of server 204. In such embodiments, processor 312 can execute at least a portion of the computer program to transmit content (e.g., one or more holograms) to one or more XR devices 100, receive content from one or more XR devices 100, receive instructions from one or more devices (e.g., XR device 100-1, input device 230, another server, a personal computer, a laptop computer, a tablet computer, a smartphone, etc.).

In some embodiments, input device 230 can include a processor 322, one or more inputs 324, one or more communication systems 326, and/or memory 328. In some embodiments, processor 322 can be any suitable hardware processor or combination of processors, such as a CPU, a GPU, an APU, etc. In some embodiments, inputs 324 can include any suitable input devices and/or sensors that can be used to receive user input, such as one or more physical or software buttons, one or movement sensors, a microphone, a touchpad, etc.

In some embodiments, communications systems 326 can include any suitable hardware, firmware, and/or software for communicating information over communications link 232, communications link 234, and/or any other suitable communications links. For example, communications systems 326 can include one or more transceivers, one or more communication chips and/or chip sets, etc. In a more particular example, communications systems 326 can include hardware, firmware and/or software that can be used to establish a Wi-Fi connection, a Bluetooth connection, a cellular connection, etc.

In some embodiments, memory 328 can include any suitable storage device or devices that can be used to store instructions, values, etc., that can be used, for example, by processor 322 to determine when input (e.g., user input) is received, to record sensor data, to communicate sensor data with one or more XR devices 100, etc. Memory 328 can include any suitable volatile memory, non-volatile memory, storage, any other suitable type of storage medium, or any suitable combination thereof. For example, memory 328 can include RAM, ROM, EEPROM, one or more flash drives, one or more hard disks, one or more solid state drives, one or more optical drives, etc. In some embodiments, memory 328 can have encoded thereon a computer program for controlling operation of input device 230. In such embodiments, processor 322 can execute at least a portion of the computer program to transmit data (e.g., representing sensor outputs) to one or more XR device 100, to transmit data (e.g., representing sensor outputs) to one or more servers 204, etc.

FIG. 4A shows an example 400 of an information flow among content source devices 204-2 and 204-2′ uploading content and/or user interface instructions to an API server 204-1, and an extended reality device receiving the content and/or user interface instructions and presenting the content in accordance with the user interface instructions in accordance with some embodiments of the disclosed subject matter.

As shown in FIG. 4A, at 402, in some embodiments, a first content source (e.g., content source 1 204-2) can provide content (e.g., 2D content, 3D content, information indicative of where an object is located with respect to an XR device and/or another object in an environment, etc.) and/or user interface instructions indicative of rules for presenting the content to API server 204-1. In some embodiments, the content can be in any suitable format, such as one or more .obj files, one or more .fbx files, one or more .gltf files, one or more .glb files, one or more data points (e.g., a position of an object with respect to an anchor point), etc.

In some embodiments, the user interface instructions can include any suitable instructions that can be used to cause an XR device (e.g., via an XR presentation application) to instantiate and/or present an object at a particular location (e.g., with respect to a particular anchor point), with a particular orientation and/or scale (e.g., with respect to a particular reference). For example, user interface instructions can include an instruction to load particular content from any suitable source, which can be local to the XR device or remote from the XR device. In such an example, the user interface instructions can include an instruction to load content provided by the content source (e.g., content provided to the XR device via the API). Additionally or alternatively, the user interface instructions can include an instruction to load content that is located at a particular location (e.g., a web address, an IP address, etc.). As another example, user interface instructions can include an instruction to generate a shape that can be constructed from one or more primitives (e.g., a cube, a sphere, etc.). In some embodiments, the user interface instructions can be provided by content source 204-2 responsive to information received from the XR device. For example, content source 204-2 can request (e.g., via a get message sent to the XR device via an API server) and/or otherwise receive information (e.g., via a push message sent by the XR device) about content that content source 204-2 caused to be generated and/or presented by the XR device. In some embodiments, the user interface instructions can include conditional statements (e.g., if user interacts with object, then perform some action). In some embodiments, the user interface instructions can indicate whether an object can be manipulated (e.g., via a flag associated with the object), how the object can be manipulated (e.g., whether the object can rotated, scaled, etc., whether the object interacts with other objects in an XR scene or whether interactions are limited to direct manipulation, for example, via a user's hand interacting with the object).

At 404, API server 204-1 can format the content and/or user interface instructions received from server 204-2 in a format that can be used by an XR presentation application to present the content and/or to control presentation of content based on the user interface instructions. For example, content source 204-2 can generate a JSON object (e.g., via an application implemented in Python) that includes a user interface instruction(s), and API server 204-1 can translate the instruction(s) in the JSON object into a core library command that can be used by the XR device to carry out the instruction(s).

At 406, API server 204-1 can provide the content and/or user interface instructions (e.g., in a suitable format) provided/received at 404 to one or more XR devices.

At 408, a second content source (e.g., content source 2 204-2′) can provide different content (e.g., 2D content, 3D content, information indicative of where an object is located with respect to an XR device and/or another object in an environment, etc.) and/or user interface instructions indicative of rules for presenting the content to API server 204-1.

At 410, API server 204-1 can format the content and/or user interface instructions received from server 204-2′ in a format that can be used by an XR presentation application to present the content and/or to control presentation of content based on the user interface instructions.

At 412, API server 204-1 can provide the content and/or user interface instructions (e.g., in a suitable format) provided/received at 408 to one or more XR devices.

At 414, XR device 100 can store content and/or user interface instructions provided/received at 406 and/or at 412. In some embodiments, XR device 100 can store content and/or user interface instructions in any suitable memory, such as cache memory. In some embodiments, XR device 100 can store content and/or user interface instructions in temporary memory (e.g., not in storage such as a solid state drive, a hard drive, flash memory, etc.), which can be cleared periodically (e.g., at regular and/or irregular intervals). Additionally or alternatively, in some embodiments, XR device 100 can receive and store content and/or user interface instructions that are encrypted, and which can be decrypted for use in presenting and/or interacting with content using a suitable decryption key.

At 416, XR device 100 can present and/or update content 1 (e.g., content received from content source 1) and/or content 2 (e.g., content received from content source 2) based on associated user interface instructions and, in some cases, environmental cues.

At 418, content source 204-2′ can request information about one or more objects presented by an XR device (e.g., via a get request provided to API server 204-1). For example, content source 204-2′ can request information about one or more objects that content source 204-2′ has caused the XR device to instantiate and/or display. In some embodiments, 418 can be omitted (e.g., in a system that implements a push model in which updates related to objects are provided to content source 204-2′ without a request first being sent by content source 204-2′).

At 420, API server 204-1 can provide transmit a request for information about one or more objects presented by XR device 100 (e.g., in response to the request transmitted at 418, periodically at regular and/or irregular intervals, etc.).

At 422, XR device 100 can provide information about one or more objects, such as a current location (e.g., with respect to a particular anchor point), a current scale, a current orientation, a current state, etc. to the content source (e.g., via API server 204-1, directly without providing it to API server 204-1, via a different API server, etc.). At 424, API server 204-1 can provide the information about one or more objects to content source 204-2′. In some embodiments, content source 204-2′ can analyze the information, and can provide updated content and/or user interface instructions at 408.

FIG. 4B shows another example 430 of an information flow among content source devices 204-2 and 204-2′ uploading content and/or user interface instructions to an API server 204-1, and an extended reality device receiving the content and/or user interface instructions and presenting the content in accordance with the user interface instructions in accordance with some embodiments of the disclosed subject matter.

As shown in FIG. 4B, at 432, in some embodiments, a content source (e.g., content source 204-2) can provide user interface instructions indicative of rules for presenting content to API server 204-1. For example, content source 204-2 can operate to control a portion of an XR presentation without being the source from which any content to be presented is sent to the XR devices participating in the presentation. In such an example, content source 204-2 can provide instructions that can be used by the XR devices to create and/or retrieve content to be presented (e.g., rather than providing the content directly to the XR devices via API server 204-1). Additionally or alternatively, in some embodiments, a user computing device can act as content source 204-2.

In some embodiments, the user interface instructions can include any suitable instructions that can be used to cause an XR device (e.g., via an XR presentation application) to instantiate and/or present an object at a particular location (e.g., with respect to a particular anchor point), with a particular orientation and/or scale (e.g., with respect to a particular reference). For example, user interface instructions can include an instruction to load particular content from any suitable source, which can be local to the XR device or remote from the XR device. In such an example, the user interface instructions can include an instruction to load content indicated by the content source (e.g., content provided to the XR device via the API, content instantiated by the XR device, and/or content provided to the XR device from a storage location referenced in instructions sent via the API). As another example, user interface instructions can include an instruction to generate a shape that can be constructed from one or more primitives (e.g., a cube, a sphere, etc.). In some embodiments, the user interface instructions can be provided by content source 204-2 responsive to information provided by an XR device. For example, content source 204-2 can request (e.g., via a get message sent to the XR device via an API server) and/or otherwise receive information (e.g., via a push message sent by the XR device) about content that content source 204-2 caused to be generated and/or presented by the XR device. In some embodiments, the user interface instructions can include conditional statements (e.g., if user interacts with object, then perform some action). In some embodiments, the user interface instructions can indicate whether an object can be manipulated (e.g., via a flag associated with the object), how the object can be manipulated (e.g., whether the object can be rotated, scaled, etc., whether the object interacts with other objects in an XR scene or whether interactions are limited to direct manipulation, for example, via a user's hand interacting with the object).

At 434, content source 204-2 can provide instructions to generate content (e.g., based on one or more primitives) and store the content and/or properties of the content at a particular storage location (e.g., defined by an IP address, a universal resource locator (URL), a blob storage resource locator, a bucket resource locator, and/or using any other suitable information). In some embodiments, in addition to, or in lieu of, storing the content and/or properties of the content at a particular storage location, content source 204-2 can provide instructions indicating that updates to the content (e.g., changes in a property based on user input and/or user interface instruction enforced rules) are to be communicated to the particular storage location, and can provide instructions indicating that the XR device is to check for updates to the content at the particular storage location.

Additionally or alternatively, at 434, content source 204-2 can provide instructions to retrieve content (e.g., 3D models, textures, etc.) from the particular storage location. In some embodiments, instructions provided at 436 can be included with user interface instructions provided at 432.

At 436, API server 204-1 can format the user interface instructions and/or instructions to generate/retrieve content received from content source 204-2 in a format that can be used by an XR presentation application to present the content and/or to control presentation of content based on the user interface instructions. For example, content source 204-2 can generate a JSON object (e.g., via an application implemented in Python) that includes a user interface instruction(s), and API server 204-1 can translate the instruction(s) in the JSON object into a core library command that can be used by the XR device to carry out the instruction(s).

At 438, API server 204-1 can provide the user interface instructions (e.g., in a suitable format) provided at 432 and/or formatted at 436 to one or more XR devices 100.

At 440, API server 204-1 can provide the instructions to generate and/or load content (e.g., in a suitable format) provided at 434 and/or formatted at 436 to one or more XR devices 100.

At 442, XR device 100 can load user interface instructions provided at 438. For example, XR device 100 can store the user interface instructions provided/received at 438. In some embodiments, XR device 100 can store user interface instructions in any suitable memory, such as cache memory. In some embodiments, XR device 100 can store user interface instructions in temporary memory (e.g., not in storage such as a solid state drive, a hard drive, flash memory, etc.), which can be cleared periodically (e.g., at regular and/or irregular intervals). Additionally or alternatively, in some embodiments, XR device 100 can receive and store user interface instructions that are encrypted, and which can be decrypted for use in presenting and/or interacting with content using a suitable decryption key. In some embodiments, although only user interface instructions from a single source are shown as being loaded in FIG. 4B, user interface instructions from multiple sources can be received (e.g., via API server 204-1) and loaded simultaneously.

At 444, XR device 100 can generate content based on instructions provided/received at 440. For example, if the instructions provided at 440 include instructions to instantiate any objects (e.g., from primitives included in a core library associated with an extended reality presentation application), XR device 100 can instantiate such objects at 444. In some embodiments, 444 can be omitted (e.g., when the user interface instructions and/or instructions provided at 440 omit any instructions to instantiate objects).

At 446, XR device 100 can request content from a storage server and/or storage service 204-2′ (referred to below as storage server 204-2′) based on instructions provided/received at 440. For example, if the instructions provided at 440 include instructions to retrieve content from a particular storage location(s), XR device 100 can request any content stored at the storage location(s) at 446.

At 448, storage service 204-2′ can provide the requested content to the requesting XR device 100 (e.g., using any suitable technique or combination of techniques). In some embodiments, the content can be in any suitable format, such as one or more .obj files, one or more .fbx files, one or more .gltf files, one or more .glb files, one or more data points (e.g., a position of an object with respect to an anchor point), etc.

In some embodiments, XR device 100 can store content provided/received at 448. In some embodiments, XR device 100 can store the content in any suitable memory, such as cache memory. In some embodiments, XR device 100 can store content in temporary memory (e.g., not in storage such as a solid state drive, a hard drive, flash memory, etc.), which can be cleared periodically (e.g., at regular and/or irregular intervals). Additionally or alternatively, in some embodiments, XR device 100 can receive and store content that is encrypted, and which can be decrypted for use in presenting and/or interacting with content using a suitable decryption key.

At 450, XR device 100 can present and/or update content (e.g., content received generated in response to instructions from content source 204-2 and/or content received from storage service 204-2′) based on associated user interface instructions and, in some cases, environmental cues.

At 452, XR device 100 can provide updates about the content being displayed in response to content changes (e.g., changes driven by user behavior, environmental cues, and/or user interface instructions) to storage service 204-2′. For example, if a user of XR device 100 manipulates an object being presented within an XR presentation, XR device 100 can provide updates about any new and/or updated properties of the object to storage service 204-2′. In some embodiments, the updates can be provided to an address associated with the object and/or can be provided with identifying information associated with the object (e.g., to an address associated with multiple objects).

At 454, XR device 100 can request information about one or more objects presented by the XR device (e.g., via a get request provided to storage service 204-2′). For example, content source 204-2′ can request information about one or more objects that content source 204-2′ has caused the XR device to instantiate and/or display. In some embodiments, 418 can be omitted (e.g., in a system that implements a push model in which updates related to objects are provided to content source 204-2′ without a request first being sent by content source 204-2′).

At 456, storage service 204-2′ can provide updates for the requested objects to XR device 100.

At 458, XR device 100 can update content in the XR presentation based o updates provided/received at 456 from storage service 204-2′/For example, XR device 100 can update properties of the content being displayed to incorporate changes driven by user input and/or user interface instruction enforced rules.

At 460, content source 204-2 can request information about one or more objects presented by an XR device from API server 204-1.

At 462, API server 204-1 can request information about one or more objects presented by the XR device from storage service 204-2′ (e.g., via a get request provided to storage service 204-2′).

At 464, storage service 204-2′ can provide updates for the requested objects to API server 204-1.

At 466, API server 204-1 can provide updates to content source 204-2.

In some embodiments, using a pull model in which XR devices receive content and/or updates directly from a storage service (or multiple storage services) can facilitate participation in an XR presentation by more XR devices than using a push model in which API server 204-1 communicates content and updates to the XR devices. For example, in a push model, the refresh rate can be limited due to the number of individual messages that are sent to different XR devices from a common API server. Additionally, a refresh rate at which the XR presentation operates may be limited by an XR device that is least capable (e.g., to synchronize the presentation, the API server can limit refreshes to a refresh rate that all participating XR devices can achieve). In a pull model, XR devices with different capabilities can operate at different refresh rates, as updates can be disseminated by a storage service (e.g., which can scale automatically to handle more requests) that can fulfill a large volume of requests relatively quickly.

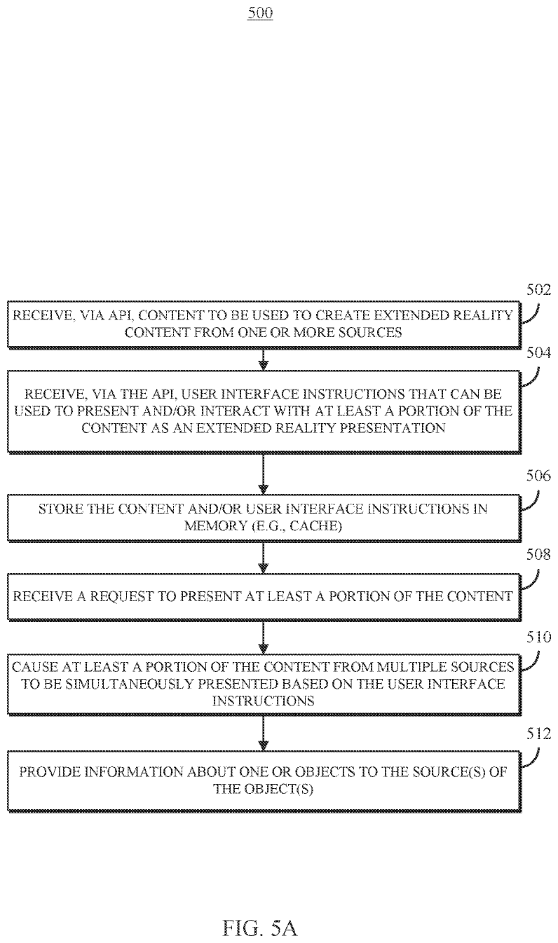

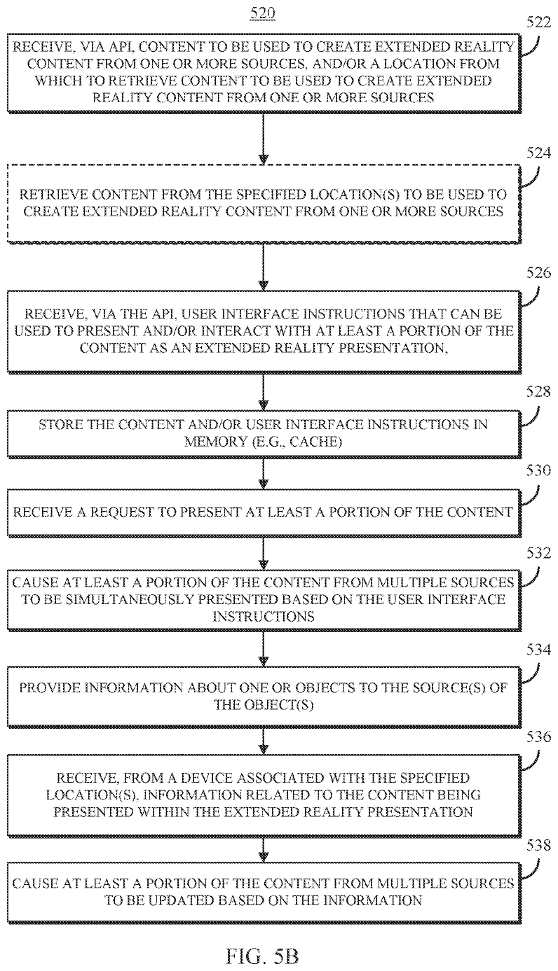

FIG. 5A shows an example 500 of a process for providing content and user interface instructions to one or more extended reality devices in accordance with some embodiments of the disclosed subject matter.

At 502, process 500 can receive, via an API, content to be used to create XR content from one or more sources of content. For example, process 500 can receive content from one or more content sources 204-2 via an API server 204-1.

At 504, process 500 can receive, via the API, user interface instructions that can be used to present and/or interact with at least a portion of the content received at 502 as part of an XR presentation.

At 506, process 500 can store the content and/or user interface instructions in memory, such as cache memory of the device executing process 500 (e.g., RAM), and/or in a cache associated with a processor (e.g., CPU cache, GPU cache, etc.).

At 508, process 500 can receive a request to present at least a portion of the content. For example, a user can provide input that causes an application associated with the content to be launched and/or executed. As another example, a user can provide input that causes a particular content source to be used to generate content to be presented to a user.

At 510, process 500 can cause at least a portion of the content from multiple sources (e.g., from two or more content sources) to be simultaneously presented in a single extended reality presentation based on the user interface instructions received at 504.

At 512, process 500 can provide information about one or more objects to the content source that caused the object(s) to be presented and/or has controlled presentation of the object, such as a current location (e.g., with respect to a particular anchor point), a current scale, a current orientation, a current state, etc. to the content source (e.g., via API server 204-1, directly without providing it to API server 204-1, via a different API server, etc.).

FIG. 5B shows another example 520 of a process for providing content and user interface instructions to one or more extended reality devices in accordance with some embodiments of the disclosed subject matter.

At 522, process 520 can receive, via an API, content to be used to create XR content from one or more sources of content. For example, process 500 can receive content from one or more content sources 204-2 via an API server 204-1.

Additionally or alternatively, at 522, process 500 can receive a location(s) (e.g., as an IP address, a URL, etc.) from which to retrieve content to be used to create XR content from one or more sources of content.

At 524, process 520 can retrieve content from the specified location(s) using any suitable technique or combination of techniques. For example, process 500 can submit a get request to the specified location. As another example, process 500 can request a particular object (e.g., identified by identifying information) from the specified location.

At 526, process 520 can receive, via the API, user interface instructions that can be used to present and/or interact with at least a portion of the content received at 522 and/or retrieved at 524 as part of an XR presentation.

At 528, process 520 can store the content and/or user interface instructions in memory, such as cache memory of the device executing process 520 (e.g., RAM), and/or in a cache associated with a processor (e.g., CPU cache, GPU cache, etc.).

At 530, process 520 can receive a request to present at least a portion of the content. For example, a user can provide input that causes an application associated with the content to be launched and/or executed. As another example, a user can provide input that causes a particular content source to be used to generate content to be presented to a user.

At 532, process 520 can cause at least a portion of the content from multiple sources (e.g., from two or more content sources) to be simultaneously presented in a single extended reality presentation based on the user interface instructions received at 526.

At 534, process 520 can provide information about one or more objects to the source of the object (e.g., a storage service, a storage server, a content source, etc.) and/or to a particular location specified for updates about that object(s). In some embodiments, the information about the one or more objects can include any suitable information, such as a current location (e.g., with respect to a particular anchor point), a current scale, a current orientation, a current state, etc. In some embodiments, process 520 can provide the information directly to the source of the object and/or to the particular location without providing it to API server 204-1.

At 536, process 520 can receive, from the source of the object and/or a service associated with the particular location, information about one or more objects being presented with the XR presentation. For example, process 500 can receive any suitable information about the object(s), such as a current location (e.g., with respect to a particular anchor point), a current scale, a current orientation, a current state, etc. In some embodiments, process 520 can receive the information directly from the source of the object and/or the server or service associated with the particular location without providing it to API server 204-1.

In some embodiments, process 520 can receive the information at 536 in response to a request. For example, process 520 can transmit a request (e.g., a get request) for the information. Additionally or alternatively, in some embodiments, process 520 can receive the information at 536 multiple times in response to a single request (e.g., at a particular rate, and/or in response to information about the objects changing).

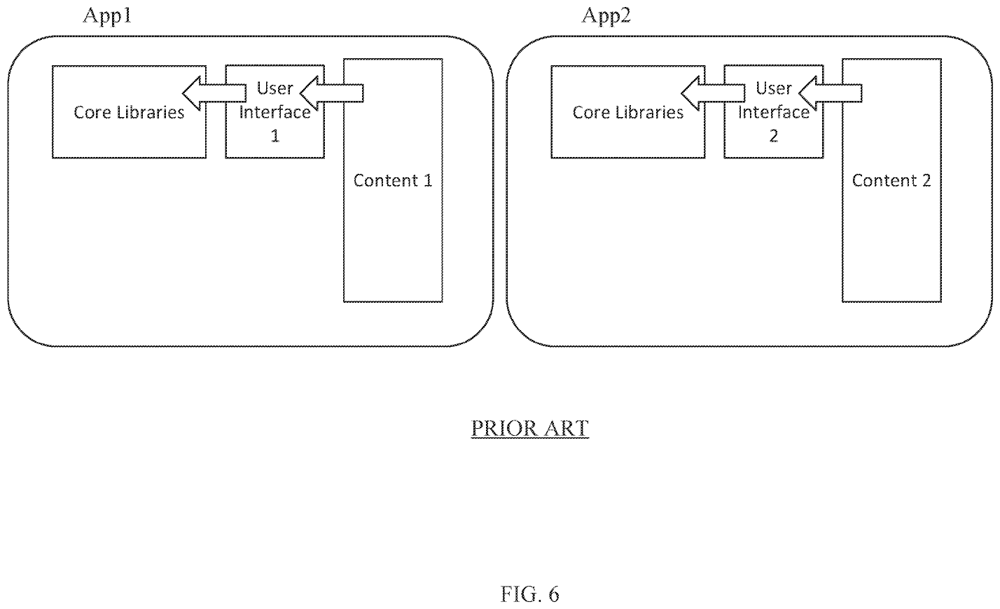

FIG. 6 shows an example of two conventional extended reality applications that include content and user interface instructions. As shown in FIG. 6, in conventional XR applications, each application is isolated, and provides a single user experience. For example, an application that is configured to present 3D anatomy content and an application that is configured to embellish a user's environment (e.g., to more closely resemble a particular landmark) generally cannot be used together, and cannot interact with one another.

In general, in order to build an application for an XR device, a developer must be able to work directly with the core libraries (e.g., Unity libraries used to cause an XR device to present 3D content as part of an XR scene, Unreal Engine libraries used to cause an XR device to present 3D content as part of an XR scene, etc.) that are used by an XR device to present XR content. For example, the developer typically must be capable of writing user interface instructions in a format that the core libraries are configured to receive.

Additionally, whenever the core libraries, the user interface, and/or the content is updated, the application must be updated to make sure that it works as expected, and must be recompiled with the updated core libraries, user interface, and/or content.

Additionally, many XR application are not built for shared experiences, and have a limited ability to network and/or interact with other XR devices that are executing the same XR application.

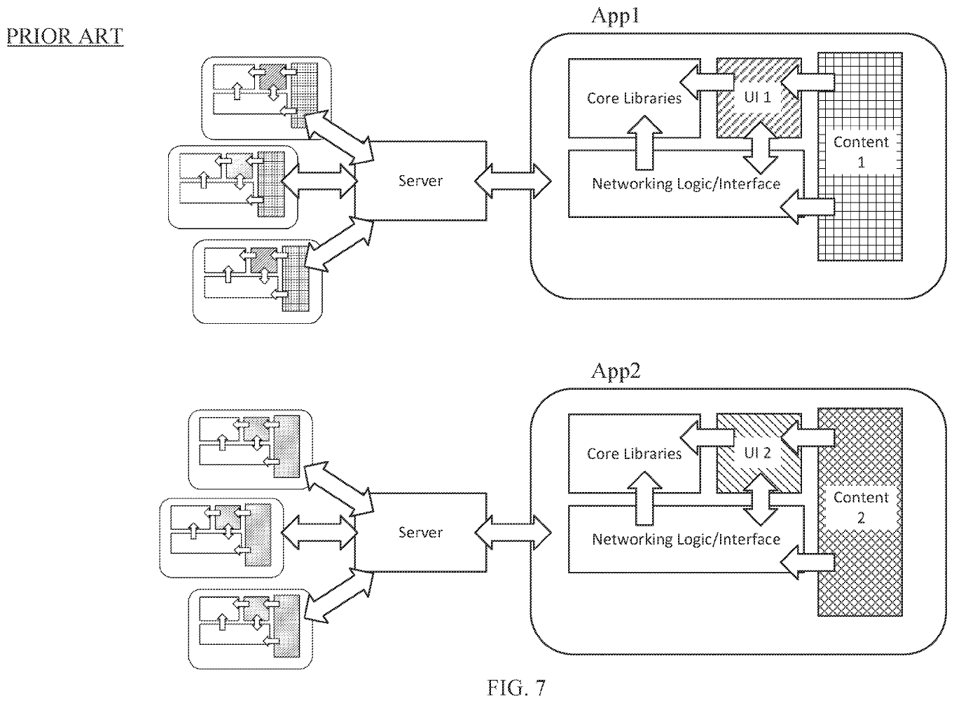

FIG. 7 shows an example, of two conventional networked extended reality applications that include content and user interface instructions used to present the content. As shown in FIG. 7, in conventional networked XR applications each application is isolated, but can be configured to provide a multi-user experience (e.g., as described in U.S. Pat. No. 10,937,391, which is hereby incorporated herein by reference in its entirety).

Each application is still isolated, and a developer still must be capable of working with the core libraries directly to build the application, and changes to the core libraries, the user interface, and/or the content require that the application be updated to insure that it works as expected, and must be recompiled with the updated core libraries, user interface, and/or content. Additionally, the networking logic must generally be customized to the UI and content used in the application, limiting efficiencies that may be expected.

In general, for the conventional applications shown in FIG. 7 to be built, the developer must have access to the content, the source code used to implement the core libraries, the UI, and the networking logic/interface elements. This can limit the ability of multiple parties to collaborate and/or can cause risks of exposing confidential information (e.g., source code of the application and/or elements of the application, such as the networking logic/interface). For example, for the conventional applications shown in FIG. 7, if a user wishes to demonstrate an application to another person, the user must either provide the other person with a device preloaded with the application, or provide the application for the other person to load (e.g., potential risking exposure of confidential information).

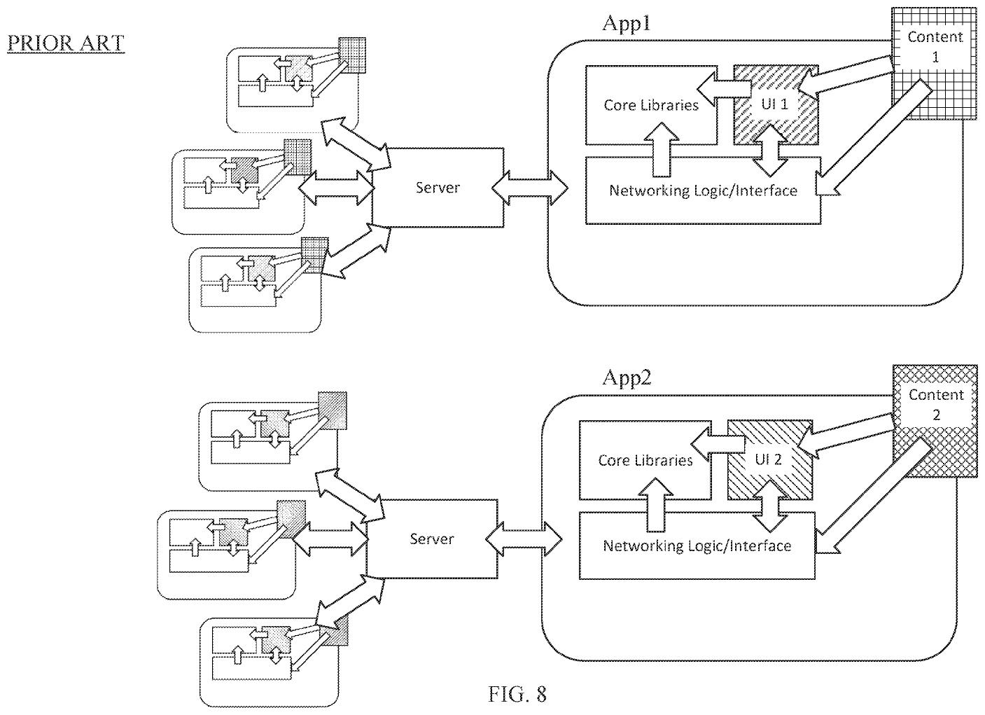

FIG. 8 shows an example of two conventional networked extended reality applications configured to dynamically load content and use included user interface instructions to present the dynamically loaded content. As shown in FIG. 8, conventional networked XR applications can be configured to dynamically load content (e.g., from a remote server). However, the UI and networking elements of the application generally must still be configured to specifically utilize the content that is going to be loaded. Such application generally cannot be used to load generic content.

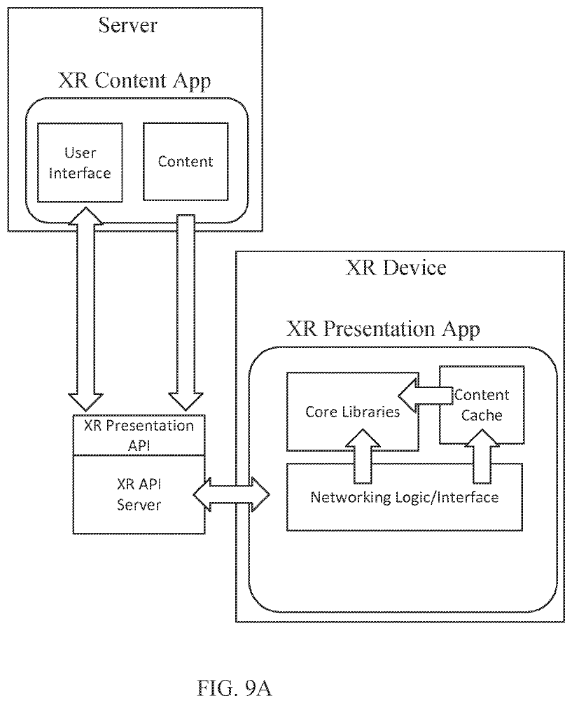

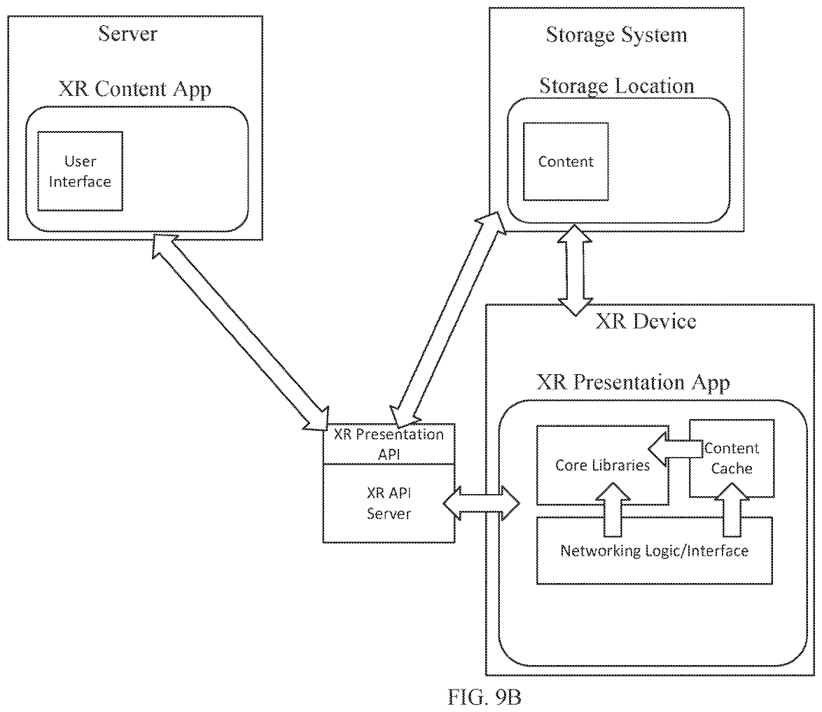

FIG. 9A shows an example of an extended reality device executing an extended reality presentation application configured to receive content and user interface instructions from an extended reality source via an extended reality presentation server and present the content based on the user interface instructions in accordance with some embodiments of the disclosed subject matter.

As shown in FIG. 9A, an XR presentation application can be executed by an XR device (e.g., XR device 100). In some embodiments, the XR presentation application can include core libraries (e.g., including code that can be used to cause the XR device to present XR content and/or content in an XR environment). In some embodiments, the core libraries can include perform some user interface tasks, such as determining where a user's hand and/or another user input device (e.g., a handheld motion controller) is located with respect to one or more XR objects in the scene (e.g., for an HMD device), whether a user has selected one or more XR objects via a user input device (e.g., a touchscreen of a smartphone or tablet computer).

In some embodiments, the XR presentation application can include and/or be associated with a cache of content that can be used to generate an XR presentation. In some embodiments, the XR presentation application can include networking logic/interface elements that can be used to receive content to be presented and/or user interface instructions to control presentation of the content.

As shown in FIG. 9A, an API server (e.g., API server 204-1) can be configured to receive content and/or user interface instructions from an XR content app (e.g., via an XR presentation API), and can provide the content and/or user interface instructions to the XR presentation application.

In some embodiments, a content server (e.g., content server 204-2) can be configured to provide content (e.g., 3D content) and/or user interface instructions to the XR device executing the XR presentation application via the XR API server.

In some embodiments, using the XR API server to provide the UI instructions and/or content from the content server, and executing an XR presentation application on the XR device can mitigate problems associated with conventional XR application development. For example, executing the XR presentation application and providing the XR API, allows a third party to build an application (e.g., XR content application) that can provide content and user interface instructions at run time (e.g., rather than providing source code to the third party, or the third party providing the content and UI to be integrated into the application ahead of time). In such an example, the XR presentation application can be updated (e.g., due to changes to the core libraries) without updating the content application, and the content application can be updated (e.g., with updated UI instructions and/or updated content) without requiring that the XR application installed on the XR device be updated with the new content and/or UI instructions prior to run time.

FIG. 9B shows another example of an extended reality device executing an extended reality presentation application configured to receive content and user interface instructions from an extended reality source via an extended reality presentation server and present the content based on the user interface instructions in accordance with some embodiments of the disclosed subject matter.

As shown in FIG. 9B, an XR presentation application can be executed by an XR device (e.g., XR device 100). As described above, the XR presentation application can include core libraries that can include perform some user interface tasks.

In some embodiments, the XR presentation application can include and/or be associated with a cache of content that can be used to generate an XR presentation. In some embodiments, the XR presentation application can include networking logic/interface elements that can be used to receive content to be presented and/or user interface instructions to control presentation of the content.

As shown in FIG. 9B, an API server (e.g., API server 204-1) can be configured to receive user interface instructions from an XR content app (e.g., via an XR presentation API), and can provide the user interface instructions to the XR presentation application.

In some embodiments, a content server (e.g., content server 204-2) can be configured to user interface instructions to the XR device executing the XR presentation application via the XR API server, and can be configured to provide instructions indicating where content (e.g., 3D content) is to be retrieved. For example, the content server can provide instructions to retrieve content from a particular location (e.g., implemented via a storage system, such as a storage server or storage service).

In some embodiments, during an XR presentation, the XR API server and/or the XR devices can communicate with the storage system to update properties of content and/or objects being presented in the XR presentation.

In some embodiments, using the XR API server to provide the UI instructions from the content server, executing an XR presentation application on the XR device, and can mitigate problems associated with conventional XR application development. For example, executing the XR presentation application and providing the XR API, allows a third party to build an application (e.g., XR content application) that can provide content and user interface instructions at run time (e.g., rather than providing source code to the third party, or the third party providing the content and UI to be integrated into the application ahead of time). In such an example, the XR presentation application can be updated (e.g., due to changes to the core libraries) without updating the content application, and the content application can be updated (e.g., with updated UI instructions and/or updated content) without requiring that the XR application installed on the XR device be updated with the new content and/or UI instructions prior to run time. Additionally, storing content and/or updates about content being presented using a storage system that has another interface (e.g., a storage API) can facilitate engagement with an XR presentation by larger number of XR devices.

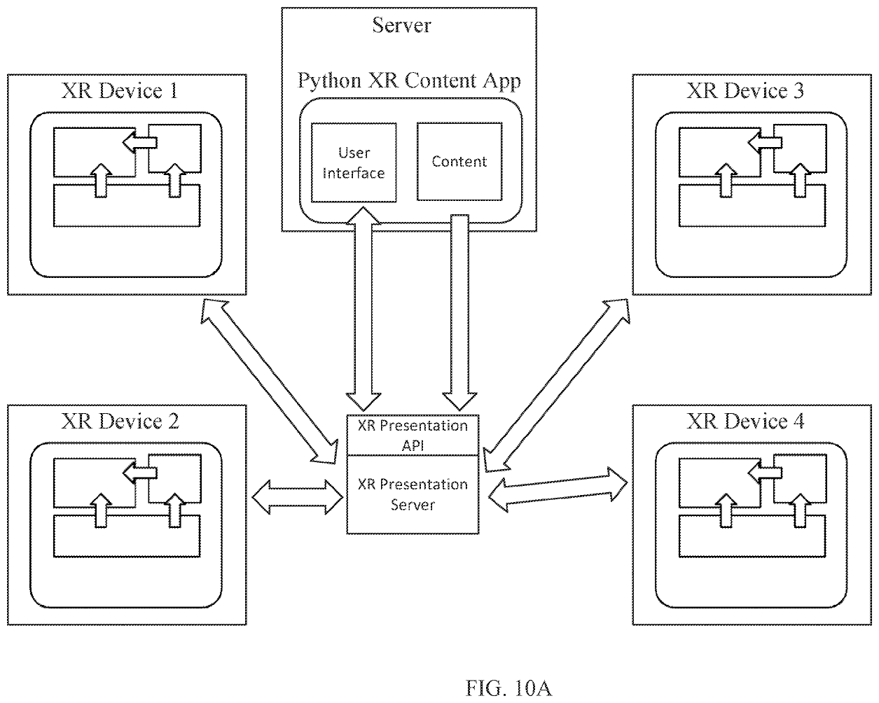

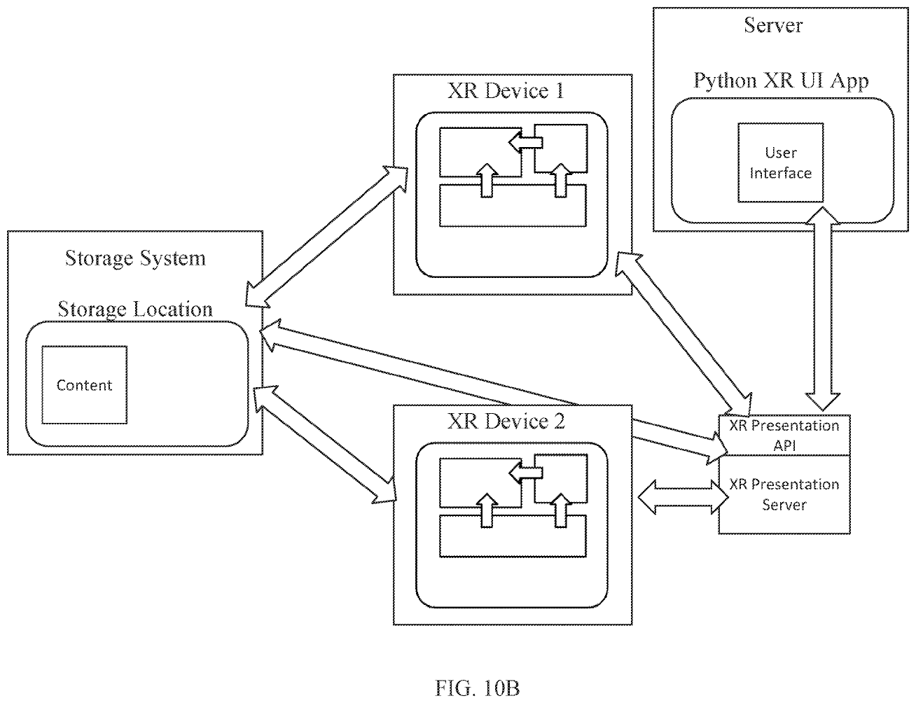

FIG. 10A shows an example of multiple extended reality devices executing extended reality presentation applications configured to receive content and user interface instructions from an extended reality source via an extended reality presentation server and present the content based on the user interface instructions in accordance with some embodiments of the disclosed subject matter.

As shown in FIG. 10A, networking can be performed by the XR presentation application installed on each device, which can facilitate group experiences without requiring a developer of the group experience to be knowledgeable of the core libraries of each XR device and/or the network logic/interface to facilitate a group experience. The XR presentation application can be configured to interface with each XR device, and can be configured to orchestrate any networking specified by the content application. This can reduce barriers to entry that limit the ability to develop group XR experiences, improving XR technology via the division of functions between an application executed by the XR device that facilitates group experiences, and an application that provides user interface rules and content. Additionally, this can allow a developer to more securely control the content by only providing the content during runtime. The content can also be associated with various security features, such as encryption, requiring that the content be stored in cache (e.g., not longer term memory), and be deleted/marked for removal after the XR experience has ended.

FIG. 10B shows an example of multiple extended reality devices executing extended reality presentation applications configured to receive content and user interface instructions from an extended reality source via an extended reality presentation server and present the content based on the user interface instructions in accordance with some embodiments of the disclosed subject matter.

As shown in FIG. 10B, networking can be performed by the XR presentation application installed on each device, which can facilitate group experiences without requiring a developer of the group experience to be knowledgeable of the core libraries of each XR device and/or the network logic/interface to facilitate a group experience. The XR presentation application can be configured to interface with each XR device, and can be configured to orchestrate any networking specified by the content application, including retrieval of content and/or updates about content from a storage system. This can reduce barriers to entry that limit the ability to develop group XR experiences, improving XR technology via the division of functions between an application executed by the XR device that facilitates group experiences, and an application that provides user interface rules and content. Additionally, this can allow a developer to more securely control the content by only providing the content during runtime. The content can also be associated with various security features, such as encryption, requiring that the content be stored in cache (e.g., not longer term memory), and be deleted/marked for removal after the XR experience has ended.

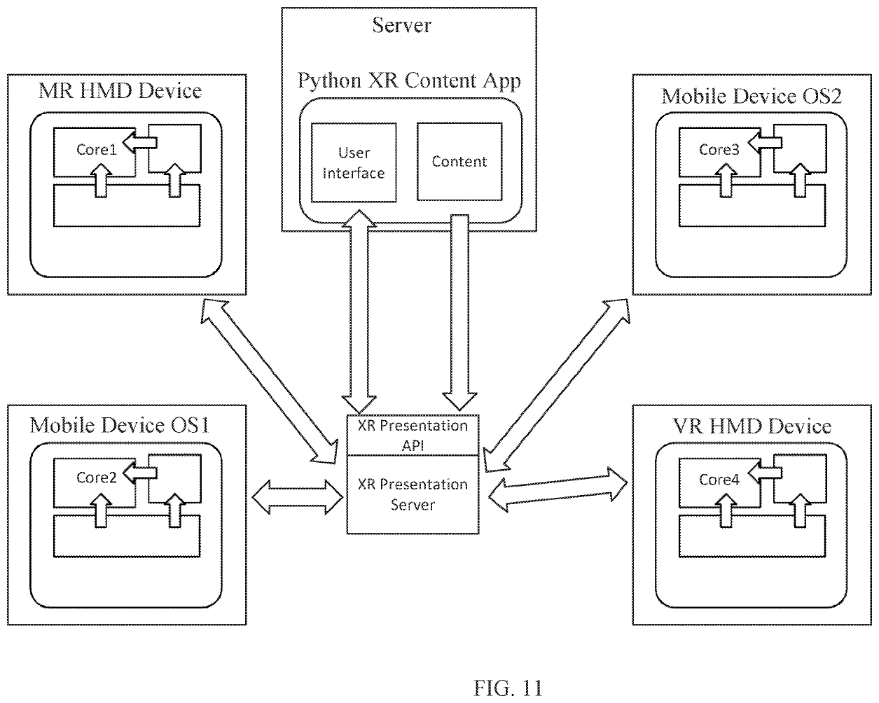

FIG. 11 shows an example of multiple different types of extended reality devices executing extended reality presentation applications configured to receive content and user interface instructions from an extended reality source via an extended reality presentation server and present the content based on the user interface instructions in accordance with some embodiments of the disclosed subject matter.

As shown in FIG. 11, different XR presentation applications can be developed for various platforms (e.g., AR/MR HMDs from different companies, mobile devices such as smartphones and tablets, VR HMDs from different companies). Each XR presentation application can use different core libraries that can facilitate presentation of XR content on a particular platform. For example, an XR presentation application configured to be executed by a mixed reality head mounted display (e.g., a HoloLens 2 HMD from Microsoft) can have a first set of core libraries (e.g., core libraries 1) configured to facilitate presentation of immersive mixed reality presentations, while a mobile device with a first operating system (e.g., iOS from Apple) can have a different set of core libraries (e.g., core libraries 2) from the MR HMD, which are configured to facilitate a less immersive MR or AR presentation. As another example, a mobile device with a second operating system (e.g., Android from Google) can have yet another different set of core libraries (e.g., core libraries 3), which are configured to facilitate a less immersive MR or AR presentation. As yet another example, a virtual reality HMD (e.g., a Meta Quest 2 from Meta Platforms) can have yet still another different set of core libraries (e.g., core libraries 4), which are configured to facilitate an immersive VR presentation. In some embodiments, mechanisms described herein can facilitate presentation of the same content on various different platforms (e.g., via platform-specific XR presentation applications) without requiring a develop of an XR experience that uses the content to develop a different application for each platform.

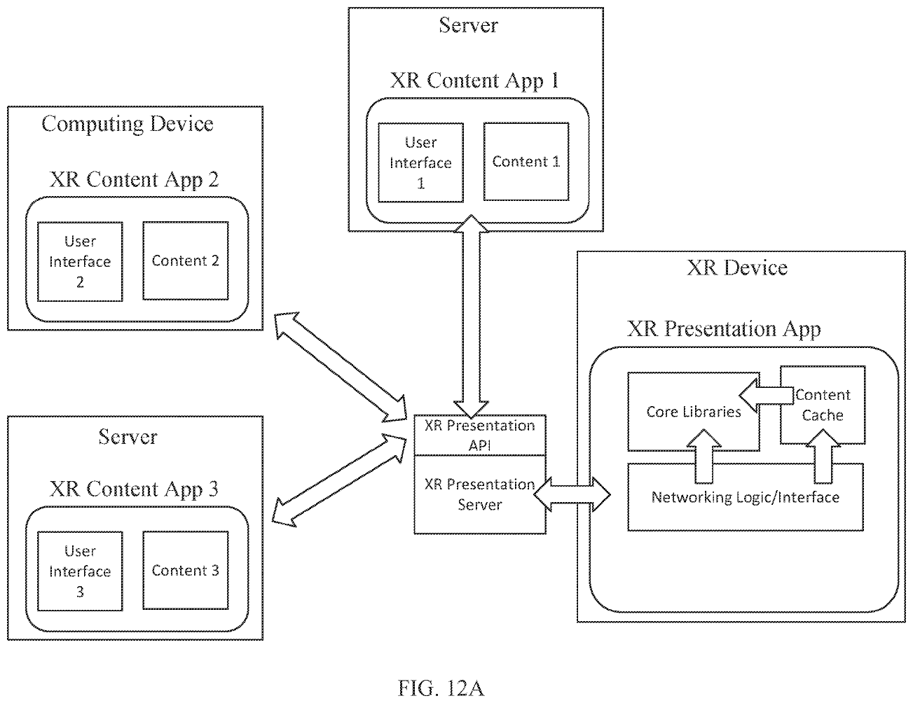

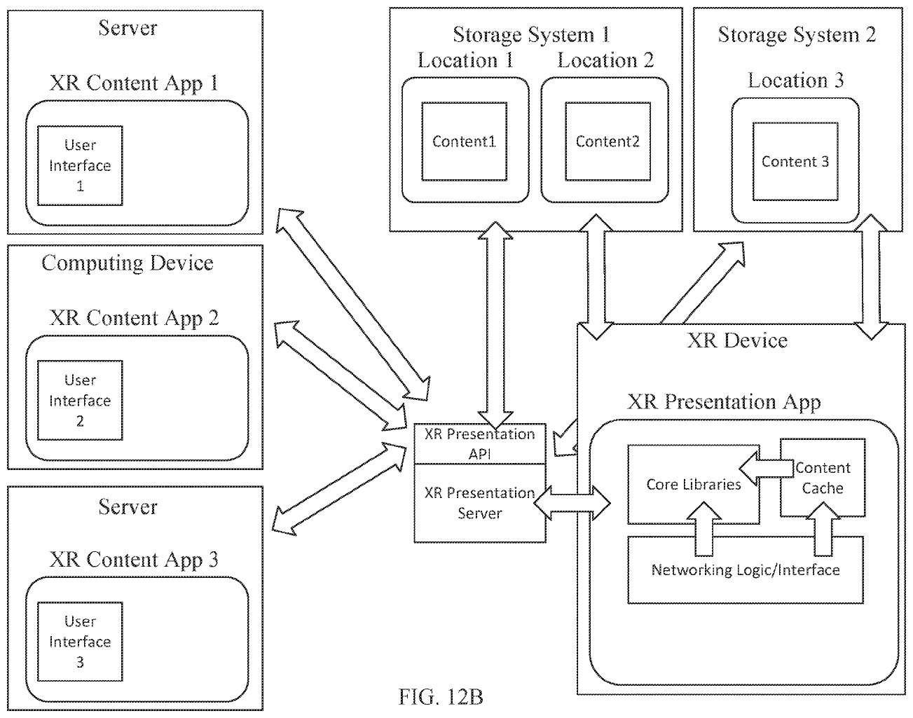

FIG. 12A shows an example of an extended reality device executing an extended reality presentation application configured to receive content and user interface instructions from extended reality sources via an extended reality presentation server, and multiple extended reality sources configured to provide content to the extended reality device in accordance with some embodiments of the disclosed subject matter. As shown in FIG. 12A, different XR content applications can be developed independently, and can be utilized simultaneously by an XR device executing an XR presentation application implemented in accordance with some embodiments of the disclosed subject matter. For example, each XR content application can provide independent UI instructions and content to the XR device via the XR API server, and the XR presentation application can present content from each application simultaneously in accordance with the UI instructions using the core libraries.

FIG. 12B shows another example of an extended reality device executing an extended reality presentation application configured to receive user interface instructions from multiple extended reality sources (e.g., via an extended reality presentation server), and multiple storage systems configured to provide content to the extended reality device in accordance with some embodiments of the disclosed subject matter. As shown in FIG. 12B, different XR content applications can be developed independently, and can be utilized simultaneously by an XR device executing an XR presentation application implemented in accordance with some embodiments of the disclosed subject matter. For example, each XR content application can provide independent UI instructions to the XR device via the XR API server, and can specify content to be retrieved by the XR device via the XR API server, and the XR presentation application can present content associated with each application (e.g., specified by each application, and retrieved from a particular location) simultaneously in accordance with the UI instructions using the core libraries. In some embodiments, content associated with different XR applications can be stored by the same storage system (e.g., Microsoft Azure Blob Storage), but may be stored separately (e.g., in connection with different accounts), and multiple storage systems can be accessed to retrieve content to be presented in a single XR presentation (e.g., certain content associated with the first and second XR applications may be stored by a first storage system, while content associated with a third XR applications may be stored by a different, second storage system).

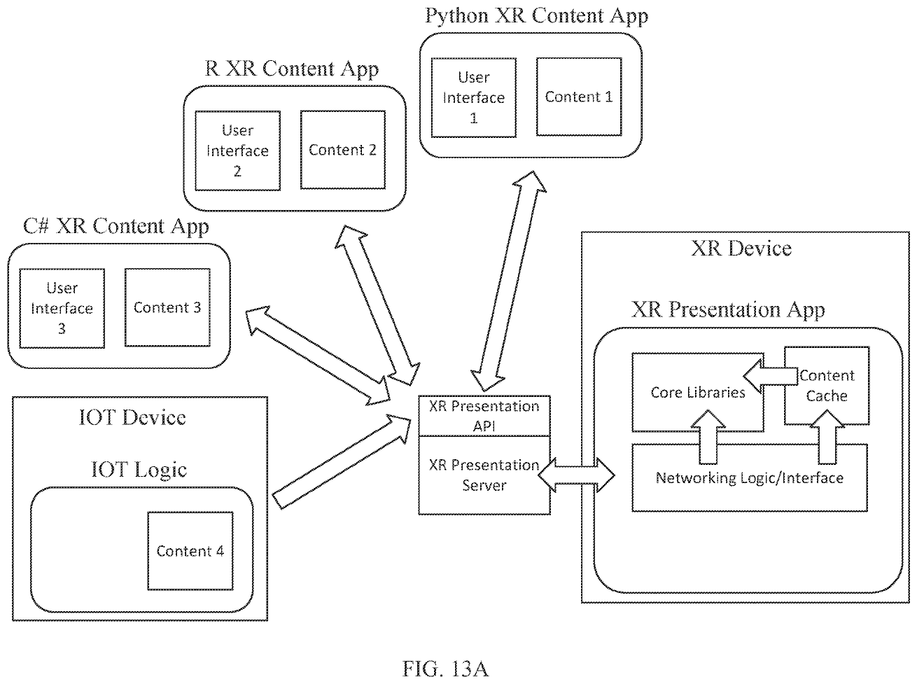

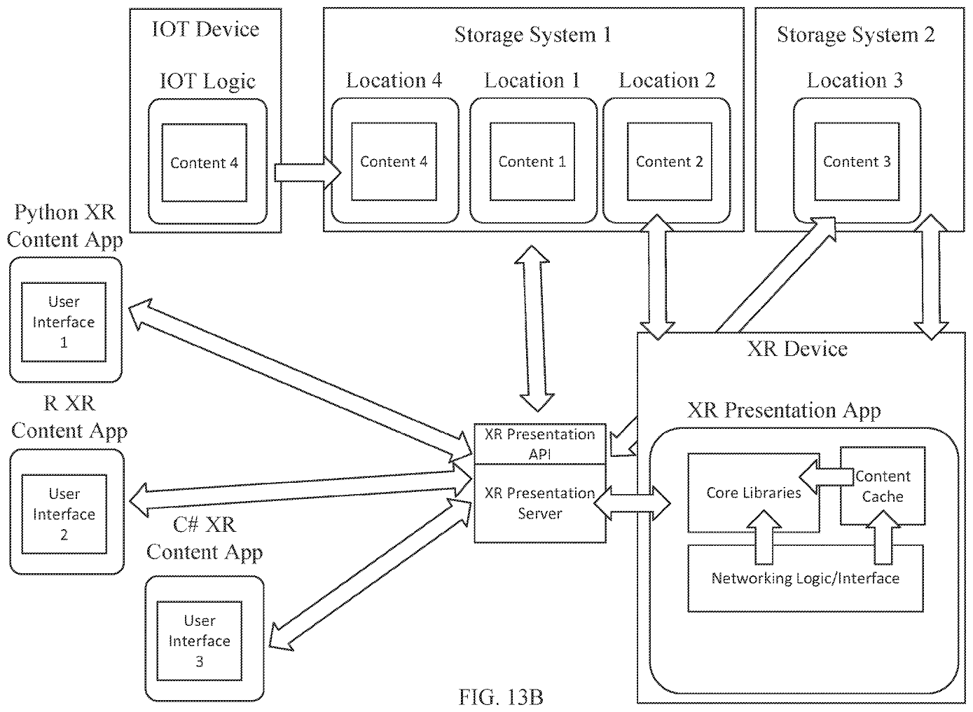

FIG. 13A shows an example of an extended reality device executing an extended reality presentation application configured to receive content and user interface instructions from extended reality sources via an extended reality presentation server, and multiple extended reality sources implemented in different programming languages configured to provide content to the extended reality device in accordance with some embodiments of the disclosed subject matter.

As shown in FIG. 13A, XR content applications can be programmed in multiple languages, and/or content can be provided by devices that are not specifically configured for XR (e.g., internet-of-things devices). For example, content and/or UI instructions specified in different languages can be provided to the XR API server, which can format the instructions and/or content for presentation by the XR presentation application. This can facilitate broader development of XR content applications without requiring developers to learn new languages and/or development platforms. For example, a developer that is proficient in Python can write an XR content application in Python, rather than attempting to learn Unity or another development platform (e.g., Unreal Engine) to develop an entire XR application. This can dramatically increase the availability of high quality XR experiences.

FIG. 13B shows another example of an extended reality device executing an extended reality presentation application configured to receive content and user interface instructions from extended reality sources via an extended reality presentation server, and multiple extended reality sources implemented in different programming languages configured to provide content to the extended reality device in accordance with some embodiments of the disclosed subject matter.

As shown in FIG. 13B, XR content applications can be programmed in multiple languages, and/or content can be provided by devices that are not specifically configured for XR (e.g., internet-of-things (IoT) devices). For example, UI instructions specified in different languages can be provided to the XR API server, which can format the instructions for presentation by the XR presentation application, and content associated with the different UI instructions can be retrieved from one or more storage systems (which can be, e.g., selected by a developer associated with the UI). This can facilitate broader development of XR content applications without requiring developers to learn new languages and/or development platforms. For example, a developer that is proficient in Python can write an XR content application in Python, rather than attempting to learn Unity or another development platform (e.g., Unreal Engine) to develop an entire XR application. This can dramatically increase the availability of high quality XR experiences. In some embodiments, data sources, such as IoT devices, can store data in a particular location, and XR devices can be instructed to retrieve data from that location in order to utilize the IoT-generated data in an XR presentation.

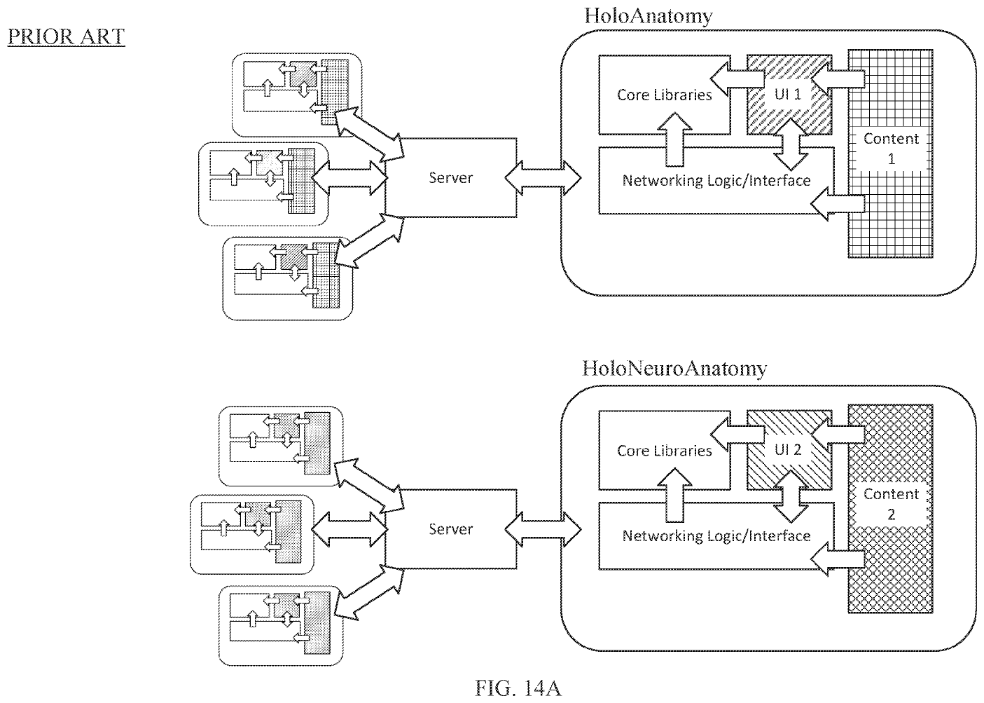

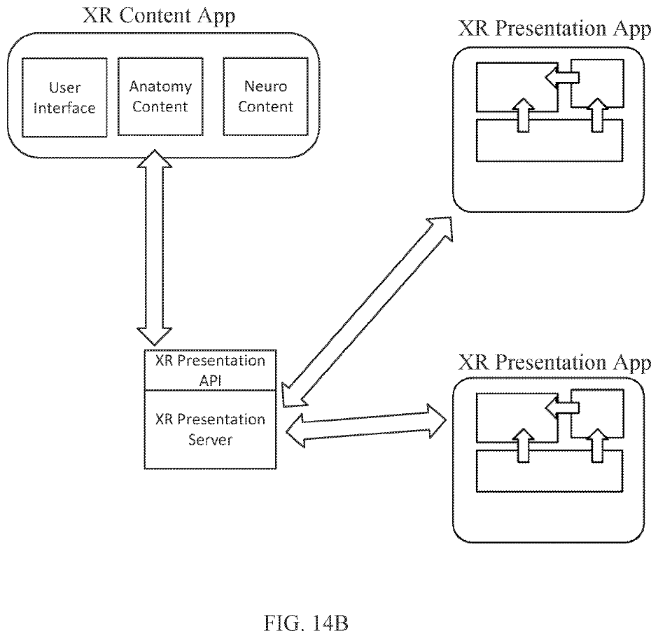

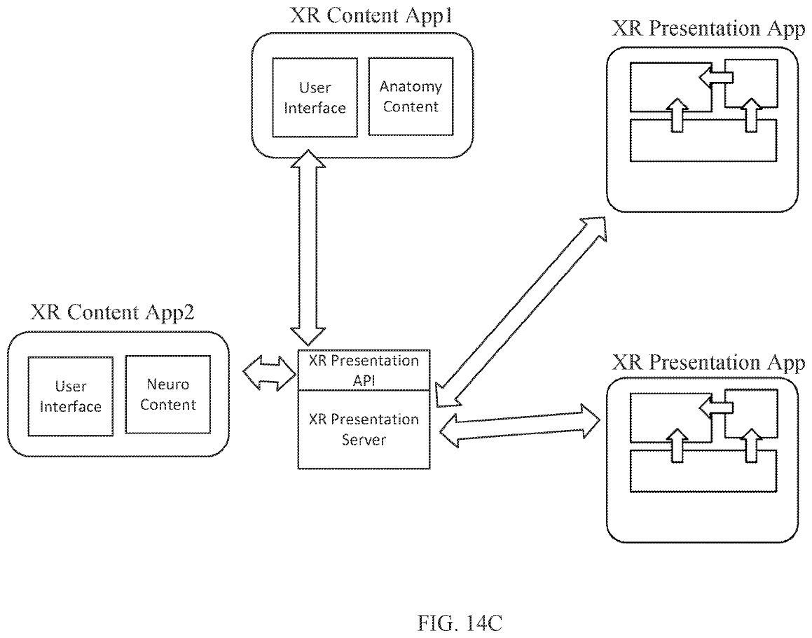

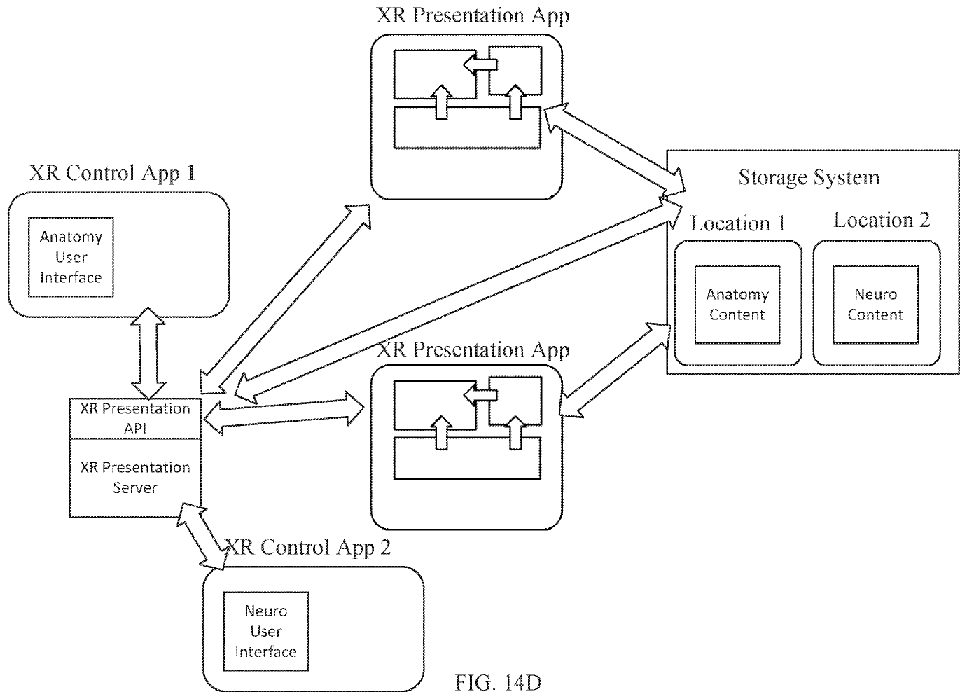

FIG. 14A shows a more particular example, of two conventional networked extended reality applications that include content and user interface instructions used to present the content. As shown in FIG. 14A, in conventional XR applications, applications can be built to present different content (e.g., general anatomy, and neurological-specific anatomy). If a developer wanted to present both sets of content in a unified XR experience, the developer would need to incorporate all of the content into a single application, and would need to update the UI to handle navigation of both sets of content.

FIG. 14B shows an example of multiple extended reality devices executing extended reality presentation applications configured to receive content and user interface instructions from an extended reality source via an extended reality presentation server, and an extended reality source configured to provide multiple sets of content to the extended reality devices in accordance with some embodiments of the disclosed subject matter. As shown in FIG. 14B, mechanisms described herein can be used to reduce the burden on a developer of integrating content from two different sets of content in a single XR experience. For example, an XR content application can be implemented that includes both sets of content.

FIG. 14C shows an example of multiple extended reality devices executing extended reality presentation applications configured to receive content and user interface instructions from an extended reality source via an extended reality presentation server, and multiple extended reality sources configured to provide different sets of content to the extended reality devices in accordance with some embodiments of the disclosed subject matter. Additionally or alternatively, two different XR content applications can be built that facilitate access to different sets of content, and both applications can be simultaneously accessed.

FIG. 14D shows another example of multiple extended reality devices executing extended reality presentation applications configured to receive content and user interface instructions from an extended reality source via an extended reality presentation server, and multiple extended reality sources configured to provide different sets of content to the extended reality devices in accordance with some embodiments of the disclosed subject matter. As shown in FIG. 14D, one or more XR control applications can provide UI instructions (e.g., via XR presentation API) for controlling different sets of content (e.g., which can be stored using any suitable storage system), which can facilitate access to different sets of content, and both applications can be simultaneously accessed and/or content associated with both applications can be simultaneously presented.

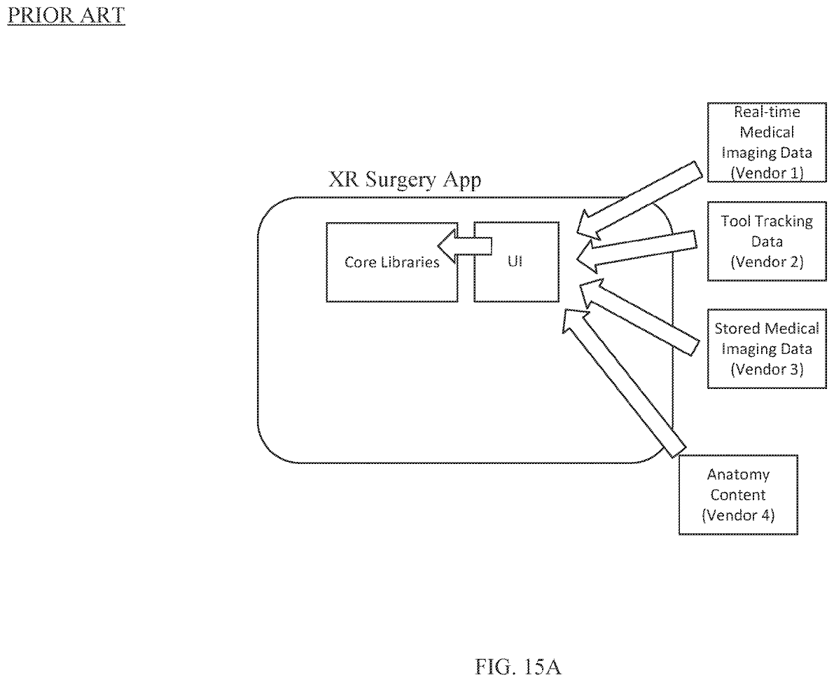

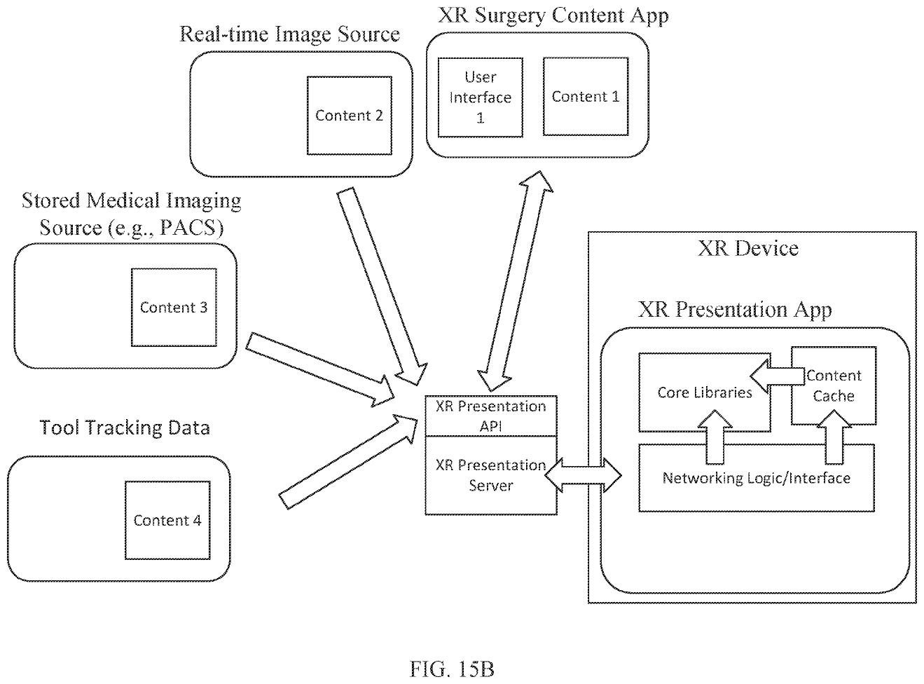

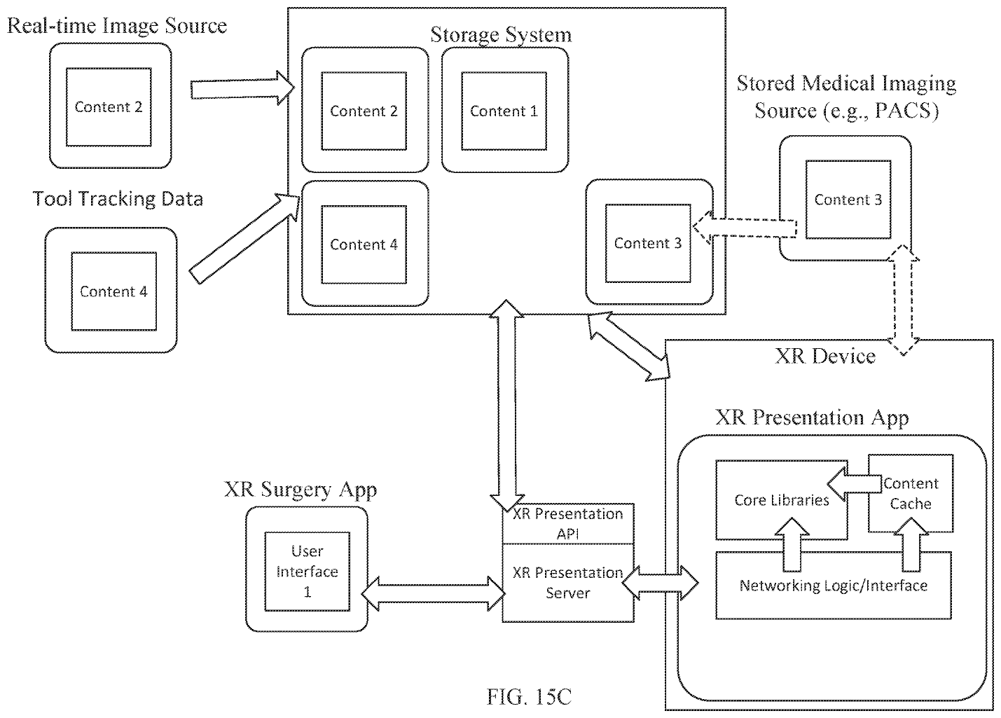

FIG. 15A shows an example of a conventional extended reality application configured to dynamically load content from multiple sources, and present extended reality content based on the multiple sources of content. In FIG. 15A, an XR surgery application can be configured to integrate data that is dynamically loaded from different sources to assist a surgeon in performing a procedure. For example, the XR surgery application can be configured to receive real-time medical imaging data (e.g., ultrasound data) from a first source (e.g., an ultrasound machine), which can be used to visualize interior structures in real-time. The XR surgery application can be configured to receive stored medical imaging data (e.g., MRI data) from another source (e.g., from a picture archiving and communication system (PACS)), which can be used to present previously obtained medical imaging data (e.g., for planning, to register the real-time medical imaging data to the subject's anatomy, etc.). The XR surgery application can be configured to receive tool tracking data (e.g., the location of a surgical tool with respect to the subject's anatomy) from yet another source, and can be configured to present anatomical data from a library of anatomical data from yet another source.

As described above in connection with FIGS. 6 and 8, the XR surgery application shown in FIG. 15A requires customization of the application to make sure that the data from each source is properly received and presented. Additionally, the application does not provide support for a group experience (e.g., a common experience between two surgeons, between a surgeon and a nurse, etc.). The application also does not provide support for participation by a remote user (e.g., a consulting surgeon).

Additionally, the application is adapted to a single platform, and is not reliant on external companies to provide a suitable interface for the data with the application. Any changes to the application, the equipment used to provide the various data requires a rebuild of the application, verification of the rebuilt application, and distribution of the updated application. This can make it very difficult to provide an application that can be used with equipment from different manufacturers (e.g., a different application may need to be built to use ultrasound from a different manufacturer's ultrasound machine).

FIG. 15B shows an example of an extended reality device executing an extended reality presentation application configured to receive content and user interface instructions from one or more extended reality sources via an extended reality presentation server, and multiple extended reality sources configured to provide content that can be dynamically loaded by the extended reality device and presented in an extended reality experience based on the multiple sources of content in accordance with some embodiments of the disclosed subject matter.

As shown in FIG. 15B, using mechanisms described herein, content from different data sources can be provided to the XR presentation via the XR API server, and user interface instructions and core content for the XR surgery application can be provided from an XR surgery content application. If one hospital uses a first type of ultrasound machine, and another hospital uses a second type of ultrasound machine, the same XR surgery application and XR presentation application can be used while altering the source that provides ultrasound data to the XR API server.

For example, the XR surgery content application can provide user interface instructions that instruct the XR presentation application to “draw a square with dimensions (X, Y) for image display” and associate the square with the name (Square1). The UI instruction can further instruct the XR presentation application to move (Square1) to position (x,y,z).

The XR surgery content application can provide user interface instructions that instruct the XR presentation application to “draw a sphere with radius (r) for tracking display” and associate the sphere with the name (Sphere1). The UI instruction can further instruct the XR presentation application to move (Sphere1) to position (x1,y1,z1).

In some embodiments, an XR content application (e.g., written in Python) associated with the ultrasound machine can provide ultrasound data (e.g., named Image A), and can provide user interface instructions that instruct the XR presentation application to “Display Image A on Square1.” Alternatively, in some embodiments, the XR surgery content application can provide user interface instructions that instruct XR presentation application to “Display Image A on Square1” and that defines Image A as image data received from a particular content source (e.g., an ultrasound machine). In such embodiments, the ultrasound machine can provide ultrasound data to the API server, and the API server can designate the ultrasound data in accordance with the scheme defined by the XR presentation application (e.g., as ImageA, with a tag indicating the source of the data, which can be used by the XR presentation application to appropriately name the data, etc.), and can provide the image data to the XR presentation application.

In some embodiments, an XR content application (e.g., written in C #) associated with an instrument tracking application can provide tracking data (e.g., a position (x,y,z)), and can provide user interface instructions that instruct the XR presentation application to “Move Sphere1 to position (x,y,z).” Alternatively, in some embodiments, the XR surgery content application can provide user interface instructions that instruct XR presentation application to “Move Sphere1 to new position (x,y,z)” and that defines the new position as a position received from the tracking application. In such embodiments, the tracking application can provide an updated position, and the API server can designate the position in accordance with the scheme defined by the XR presentation application (e.g., as an updated position, with a tag indicating the source of the data, which can be used by the XR presentation application to appropriately name/use the data, etc.), and can provide the position data to the XR presentation application.

FIG. 15C shows another example of an extended reality device executing an extended reality presentation application configured to receive content and user interface instructions from one or more extended reality sources via an extended reality presentation server, and multiple extended reality sources configured to provide content that can be dynamically loaded by the extended reality device and presented in an extended reality experience based on the multiple sources of content in accordance with some embodiments of the disclosed subject matter.

As shown in FIG. 15C, using mechanisms described herein, content from different data sources can be provided to the XR presentation without going through the XR API server, and user interface instructions and core content for the XR surgery application can be provided from an XR surgery content application via the XR API server. If one hospital uses a first type of ultrasound machine, and another hospital uses a second type of ultrasound machine, the same XR surgery application and XR presentation application can be used while altering the source that provides ultrasound data to the XR API server.

For example, the XR surgery content application can provide user interface instructions that instruct the XR presentation application to “draw a square with dimensions (X, Y) for image display” and associate the square with the name (Square1). The UI instruction can further instruct the XR presentation application to move (Square1) to position (x,y,z).

The XR surgery content application can provide user interface instructions that instruct the XR presentation application to “draw a sphere with radius (r) for tracking display” and associate the sphere with the name (Sphere1). The UI instruction can further instruct the XR presentation application to move (Sphere1) to position (x1,y1,z1).

In some embodiments, an XR application (e.g., written in Python) associated with the ultrasound machine can provide ultrasound data (e.g., named Image A) to a particular server and/or storage system (e.g., which can be on a local area network to which the ultrasound machine is connection, or remote from such as local area network), and can provide user interface instructions that instruct the XR presentation application to retrieve Image A from the server/storage system and to “Display Image A on Square1.” Alternatively, in some embodiments, the XR surgery content application can provide user interface instructions that instruct XR presentation application to “Display Image A on Square1” and that defines Image A as image data retrieved from a particular storage location (e.g., a storage location to which the ultrasound machine is transmitting ultrasound data). In such embodiments, the ultrasound machine can provide ultrasound data to the server/storage system, and the server/storage system can designate the ultrasound data in accordance with the scheme defined by the XR presentation application (e.g., as ImageA, with a tag indicating the source of the data, which can be used by the XR presentation application to appropriately name the data, etc.), and the XR presentation application can receive the image data from the server/storage system.

In some embodiments, an XR application (e.g., written in C #) associated with an instrument tracking application can provide tracking data (e.g., a position (x,y,z)) to the storage system, and can provide user interface instructions that instruct the XR presentation application to “Move Sphere1 to position (x,y,z).” Alternatively, in some embodiments, the XR surgery content application can provide user interface instructions that instruct XR presentation application to “Move Sphere1 to new position (x,y,z)” and that defines the new position as a position received from the tracking application. In such embodiments, the tracking application can provide an updated position, and the position can be updated in the XR presentation application in accordance with the scheme defined by the XR presentation application (e.g., as an updated position, with a tag indicating the source of the data, which can be used by the XR presentation application to appropriately name/use the data, etc.) upon receiving the updated information from the server/storage system.

### Further Examples Having a Variety of Features

Implementation examples are described in the following numbered clauses:

1. A method for presenting extended reality presentations, comprising: receiving, via an application program interface (API) server, first content from a first content source; receive, via the API server, second content from a second content source; receive, via the API server, user interface instructions from the second content source; and simultaneously present the first content and the second content in an extended reality presentation in accordance with the user interface instructions.

2. The method of clause 1, further comprising: receiving, via the API server, user interface instructions from the first content source; and presenting the first content in the extended reality presentation in accordance with the user interface instructions received from the first content source.

3. The method of any one of clauses 1 or 2, further comprising: receiving, from the API server, a request for information about one or more objects presented in the extended reality presentation; and in response to the request, providing updated information about the one or more objects to the API server.

4. The method of any one of clauses 1 to 3, wherein the first content source is associated with a first programming language, and the second content source is associated with a different, second programming language.

5. The method of any one of clauses 1 to 4, further comprising: receiving, from the API server, updated information about one or more objects presented in the extended reality presentation, wherein at least a first object of the one or more objects is associated with the first content, and a second object of the one or more objects is associated with the second content; and updating the extended reality presentation in accordance with the user interface instructions and the updated information.

6. The method of any one of clauses 1 to 5, wherein the first content source comprises a real-time imaging source, and the second content source comprises an extended reality application configured to facilitate surgery using data from the real-time imaging source.

7. A method for presenting extended reality presentations, comprising: receiving, from a first content source via an application program interface (API) server, instructions to retrieve first content from a first storage location; receiving, from a second content source via the API server, instructions to retrieve second content from a second storage location; receiving, via the API server, user interface instructions from the second content source; receiving, from the first storage location, the first content; receiving, from the second storage location, the second content; and simultaneously presenting the first content and the second content in an extended reality presentation in accordance with the user interface instructions.

8. The method of clause 7, further comprising: receiving, via the API server, user interface instructions from the first content source; and presenting the first content in the extended reality presentation in accordance with the user interface instructions received from the first content source.

9. The method of any one of clauses 7 or 8, further comprising: providing updated information about one or more objects presented in the extended reality presentation to the second storage location.

10. The method of any one of clauses 7 to 9, wherein the first content source is associated with a first programming language, and the second content source is associated with a different, second programming language.

11. The method of any one of clauses 7 to 10, further comprising: receiving, from a computing device associated with the second storage location, updated information about one or more objects presented in the extended reality presentation; and updating the extended reality presentation in accordance with the user interface instructions and the updated information.

12. The method of any one of clauses 7 to 11, wherein the first content source comprises a real-time imaging source, and the second content source comprises an extended reality application configured to facilitate surgery using data from the real-time imaging source.

13. The method of any one of clauses 7 to 12, wherein the first storage location is associated with a cloud storage service.

14. The method of clause 13, wherein the second storage location is associated with a different, second cloud storage service.

15. The method of any one of clauses 7 to 14, further comprising: requesting, from a computing device associated with the first storage location, updated information about one or more objects presented in the extended reality presentation; requesting, from a computing device associated with the second storage location, updated information about one or more objects presented in the extended reality presentation; and updating the extended reality presentation in accordance with the user interface instructions and the updated information.

16. A non-transitory computer-readable medium storing computer-executable code, comprising code for causing a computer to cause a processor to: perform a method of any of clauses 1 to 15.

17. A system for simulating interactions with an infant, comprising: at least one processor that is configured to: perform a method of any of clauses 1 to 15.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, any other suitable media that is fleeting and devoid of any semblance of permanence during transmission, and/or any suitable intangible media.

It will be appreciated by those skilled in the art that while the disclosed subject matter has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is hereby incorporated by reference, as if each such patent or publication were individually incorporated by reference herein.

Various features and advantages of the invention are set forth in the following claims.

1. A system for presenting extended reality presentations, comprising:
a transparent display;
a plurality of sensors; and
at least one processor, wherein the at least one processor is programmed to:
receive, via an application program interface (API) server, first content from a first content source;
receive, via the API server, second content from a second content source;
receive, via the API server, user interface instructions from the second content source; and
simultaneously present, via the transparent display based on data generated by the plurality of sensors, the first content and the second content in an extended reality presentation in accordance with the user interface instructions.

2. The system of claim 1, wherein the at least one processor is further programmed to:
receive, via the API server, user interface instructions from the first content source; and
present, via the transparent display, the first content in the extended reality presentation in accordance with the user interface instructions received from the first content source.

3. The system of claim 1, wherein the at least one processor is further programmed to:
receive, from the API server, a request for information about one or more objects presented in the extended reality presentation; and
in response to the request, provide updated information about the one or more objects to the API server.

4. The system of claim 1, wherein the first content source is associated with a first programming language, and the second content source is associated with a different, second programming language.

5. The system of claim 1, wherein the at least one processor is further programmed to:
receive, from the API server, updated information about one or more objects presented in the extended reality presentation, wherein at least a first object of the one or more objects is associated with the first content, and a second object of the one or more objects is associated with the second content; and
update the extended reality presentation in accordance with the user interface instructions and the updated information.

6. The system of claim 1, wherein the first content source comprises a real-time imaging source, and the second content source comprises an extended reality application configured to facilitate surgery using data from the real-time imaging source.

7-15. (canceled)

16. A method for presenting extended reality presentations, comprising:
receiving, via an application program interface (API) server, first content from a first content source;
receiving, via the API server, second content from a second content source;
receiving, via the API server, user interface instructions from the second content source; and
simultaneously presenting, via a transparent display, the first content and the second content in an extended reality presentation in accordance with the user interface instructions.

17. The method of claim 16, further comprising:
receiving, via the API server, user interface instructions from the first content source; and
presenting, via the transparent display, the first content in the extended reality presentation in accordance with the user interface instructions received from the first content source.

18. The method of claim 16, further comprising:
receiving, from the API server, a request for information about one or more objects presented in the extended reality presentation; and
in response to the request, providing updated information about the one or more objects to the API server.

19. The method of claim 16, wherein the first content source is associated with a first programming language, and the second content source is associated with a different, second programming language.

20. The method of claim 16, further comprising:
receiving, from the API server, updated information about one or more objects presented in the extended reality presentation, wherein at least a first object of the one or more objects is associated with the first content, and a second object of the one or more objects is associated with the second content; and
updating the extended reality presentation in accordance with the user interface instructions and the updated information.

21. The method of claim 16, wherein the first content source comprises a real-time imaging source, and the second content source comprises an extended reality application configured to facilitate surgery using data from the real-time imaging source.

22-31. (canceled)

32. A non-transitory computer-readable storage medium having instructions stored thereon that, when executed by processing circuitry, cause the processing circuitry to:
receive, via an application program interface (API) server, first content from a first content source;
receive, via the API server, second content from a second content source;
receive, via the API server, user interface instructions from the second content source; and
simultaneously present, via a transparent display, the first content and the second content in an extended reality presentation in accordance with the user interface instructions.

33. The non-transitory computer-readable storage medium of claim 32, wherein the instructions, when executed by the processing circuitry, cause the processing circuitry to:
receive, via the API server, user interface instructions from the first content source; and
present, via the transparent display, the first content in the extended reality presentation in accordance with the user interface instructions received from the first content source.

34. The non-transitory computer-readable storage medium of claim 32, wherein the instructions, when executed by the processing circuitry, cause the processing circuitry to:
receive, via the API server, a request for information about one or more objects presented in the extended reality presentation; and
in response to the request, provide updated information about the one or more objects to the API server.

35. The non-transitory computer-readable storage medium of claim 32, wherein the first content source is associated with a first programming language, and the second content source is associated with a different, second programming language.

36. The non-transitory computer-readable storage medium of claim 32, wherein the first content source comprises a real-time imaging source, and the second content source comprises an extended reality application configured to facilitate surgery using data from the real-time imaging source.

37. The system of claim 1, wherein the transparent display, the plurality of sensors, and the at least one processor are provided on a head mounted display device.

38. The system of claim 37, wherein the API server is provided locally on the head mounted display device.

39. The system of claim 37, wherein the API server is provided separately from the head mounted display device.
